(19) United States
(12) Patent Application Publication
(10) Pub. No.: US 2024/0002339 A1
(43) Pub. Date: Jan. 4, 2024

(54) THERAPEUTIC COMPOUNDS AND METHODS

(71) Applicants: Regents of the University of Minnesota, Minneapolis, MN (US); Moffitt Cancer Center, Tampa, FL (US)

(72) Inventors: Ingrid Gunda Georg, Minneapolis, MN (US); Timothy Ward, Minneapolis, MN (US); Erik Faber, Minneapolis, MN (US); Jon E. Hawkinson, Minneapolis, MN (US); Nan Wang, Minneapolis, MN (US); Ernst Schonbrunn, Tampa, FL (US)

(21) Appl. No.: 18/216,497

(22) Filed: Jun. 29, 2023

Related U.S. Application Data
(60) Provisional application No. 63/357,473, filed on Jun. 30, 2022.

(51) Int. Cl. C07D 209/20; C07D 403/12; C07D 405/12

(52) U.S. Cl. CPC C07D 209/20; C07D 403/12; C07D 405/12

(57) ABSTRACT

The invention provides a compound of Formula (I):

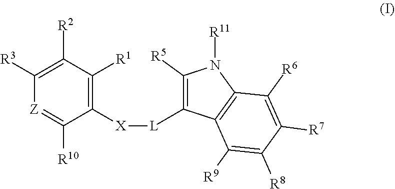

or a salt thereof, wherein R¹-R³, R⁵-R¹¹, Z, X, and L have any of the values described in the specification, as well as compositions comprising a compound of Formula (I). The compounds are useful as cyclin-dependent kinase 2 (CDK2) inhibitors.

### PRIORITY

This application claims priority to U.S. Provisional Patent Application No. 63/357,473, filed 30 Jun. 2022. The entire content of this United States Provisional Patent Applications is hereby incorporated by reference herein.

### STATEMENT REGARDING FEDERALLY SPONSORED RESEARCH

This invention was made with government support under DE-AC02-06CH11357 awarded by the U.S. Department of Energy, AGM-12006 awarded by the Other Agency, and HD099743, OD012289, CA076292, CA246363, HD080431, GM138396, GM008244, GM132029, and CA232303 awarded by the National Institutes of Health. The government has certain rights in the invention.

### BACKGROUND

Despite the attractiveness of kinases as therapeutic targets, selectively targeting a particular kinase has been historically challenging due to the structural homology of the ATP-binding site where most inhibitors bind. In contrast, inhibitors that bind an allosteric site remain an attractive way to develop selective kinase inhibitors due to the theoretical divergence of structural homology at these distal sites. Prior allosteric kinase inhibitors have even demonstrated selectivity for oncologic mutant isoforms of the same kinase (Jia, Y, et al., Nature 2016, 534 (7605), 129-32; and To, C., et al., Cancer Discov. 2019, 9 (7), 926-43).

Despite their promise, relatively few examples of high affinity allosteric kinase inhibitors exist in the literature. Cyclin-dependent kinase 2 (CDK2) is a target for both anticancer and non-hormonal contraceptive indications but an inhibitor against this kinase with exquisite selectivity has been elusive due to the structural homology between like kinases (Roskoski, R. Jr., et al., Pharmacol Res 2019, 139, 471-88; and Wood, D. J., et al., Cell Chem Biol 2018, 26, 1-10, Faber et al. 2020, ACS Chem Biol 2020, 15 (7), 1759-64).

Currently there is a need for therapeutic agents that target CDK2. In particular, there is a need for agents that are allosteric inhibitors of CDK2. Such agents would be useful, for example, as anticancer agents or as non-hormonal contraceptive agents.

### SUMMARY

The invention provides a series of allosteric inhibitors of cyclin-dependent kinase 2 (CDK2) in a type III fashion. Representative compounds bind an allosteric pocket within CDK2, as confirmed by X-ray crystallography, and demonstrate negative cooperativity with cyclin binding. This cooperative relationship is a novel mechanism of action for CDK2 inhibition.

Accordingly, in one embodiment, the invention provides a compound of the invention, which is a compound of Formula (I):

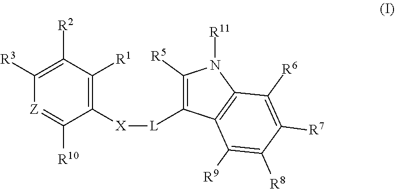

or a salt thereof, wherein:
R¹ is —COOR^{a} or tetrazol-5-yl;
R² is H, halo, nitro, trifluoromethyl, cyano, amino, (C₁-C₄)alkyl, or hydroxy;
R³ is H, nitro, cyano, halo, —SCF₃, —SF₅, (C₁-C₄)alkyl, (C₃-C₆)cycloalkyl, (C₁-C₄)alkoxycarbonyl, (C₁-C₄)alkanoyl, (C₁-C₄)alkoxy, phenyl, (C₁-C₄)alkylthio, trifluoromethyl, or trifluoromethoxy; or R³ and R^{x} taken together with the atoms to which they are attached form a 5-membered carboocycle, 6-membered carbocycle, a 5-membered heterocycle, a 6-membered heterocycle, or phenyl ring;
R⁵ is H, (C₁-C₄)alkyl, pyridyl, carboxy, or hydroxymethyl;
R⁶ is H, halo, (C₁-C₄)alkyl, or hydroxy;
R⁷ is H, halo, cyano, (C₁-C₄)alkyl, hydroxy, or (C₁-C₄)alkoxy, wherein any (C₁-C₄)alkyl and (C₁-C₄)alkoxy is optionally substituted with one or more fluoro;
R⁸ is H, halo, (C₁-C₄)alkyl, nitro, or cyano;
R⁹ is H, halo, hydroxy, or (C₁-C₄)alkyl;
R¹⁰ is H, halo, nitro, or (C₁-C₄)alkyl;
R¹¹ is H or (C₁-C₄)alkyl;
Z is CR^{x} or N;
R^{a} is H or (C₁-C₆)alkyl;
R^{x} is H, halo, nitro, (C₁-C₄)alkyl, amino, or (C₁-C₄)alkoxy; or R^{x} and R³ taken together with the atoms to which they are attached form a 5-membered carboocycle, 6-membered carbocycle, a 5-membered heterocycle, a 6-membered heterocycle, or phenyl ring;
X is S, O, or NH; and
L is absent or is (C₁-C₄)alkylene.

The invention also provides a pharmaceutical composition comprising a compound of Formula (I) or a pharmaceutically acceptable salt thereof, and a pharmaceutically acceptable excipient.

The invention also provides a method for treating cancer in an animal (e.g., a mammal such as a human) comprising, administering a compound of Formula (I) or a pharmaceutically acceptable salt thereof to the animal.

The invention also provides a compound of Formula (I) or a pharmaceutically acceptable salt thereof for use in medical therapy.

The invention also provides a compound of Formula (I) or a pharmaceutically acceptable salt thereof for the prophylactic or therapeutic treatment of cancer.

The invention also provides the use of a compound of Formula (I) or a pharmaceutically acceptable salt thereof to prepare a medicament for treating cancer in an animal (e.g. a mammal such as a human).

The invention also provides a method for producing a contraceptive effect in an animal (e.g., a mammal such as a human) comprising, administering a compound of Formula (I) or a pharmaceutically acceptable salt thereof to the animal.

The invention also provides a compound of Formula (I) or a pharmaceutically acceptable salt thereof for producing a contraceptive effect.

The invention also provides the use of a compound of Formula (I) or a pharmaceutically acceptable salt thereof to prepare a medicament for producing a contraceptive effect in an animal (e.g. a mammal such as a human).

The invention also provides a kit comprising, packaging material that contains a compound of formula (I) or a pharmaceutically acceptable salt thereof, and instructions for use of the compound of formula (I) or a pharmaceutically acceptable salt thereof as a contraceptive (e.g., to reduce sperm count in a male subject, to produce reversible infertility in a male subject, and/or to reduce the likelihood of conception or eliminate the likelihood of conception).

The invention also provides a kit comprising, packaging material that contains a compound of formula (I) or a pharmaceutically acceptable salt thereof, and instructions for use of the compound or pharmaceutically acceptable salt thereof to treat cancer.

The invention also provides a method to produce a therapeutically beneficial effect (e.g. to treat cancer or to produce a contraceptive effect) in an animal (e.g., a human) by administering to the animal, a compound that binds to an allosteric pocket within CDK2 and thereby inhibits the activity of CDK2 in the animal.

The invention also provides processes and intermediates disclosed herein that are useful for preparing a compound of Formula (I) or a salt thereof.

### BRIEF DESCRIPTION OF THE DRAWINGS

FIGS. 1A-C. Show ITC traces of Example 35 (A, K_{d}=31 nM), Example 36 (B, K_{d}=18 nM), and Example 30 (C, K_{d}=23 nM) into CDK2.

FIG. 2. Show that Example 35 and Example 14 inhibit the enzymatic activity of CDK2/cyclin A. pCDK2 (2.5 nM) was pre-incubated with the compounds for 10 minutes, followed by cyclin A (2.5 nM) addition and incubation for 30 minutes in this ADP-Glo enzymatic activity assay. Calculated IC₅₀ values are 7.0, 88, and 3400 nM for the ATP site inhibitor reference compound dinaciclib, Example 35, and Example 14, respectively.

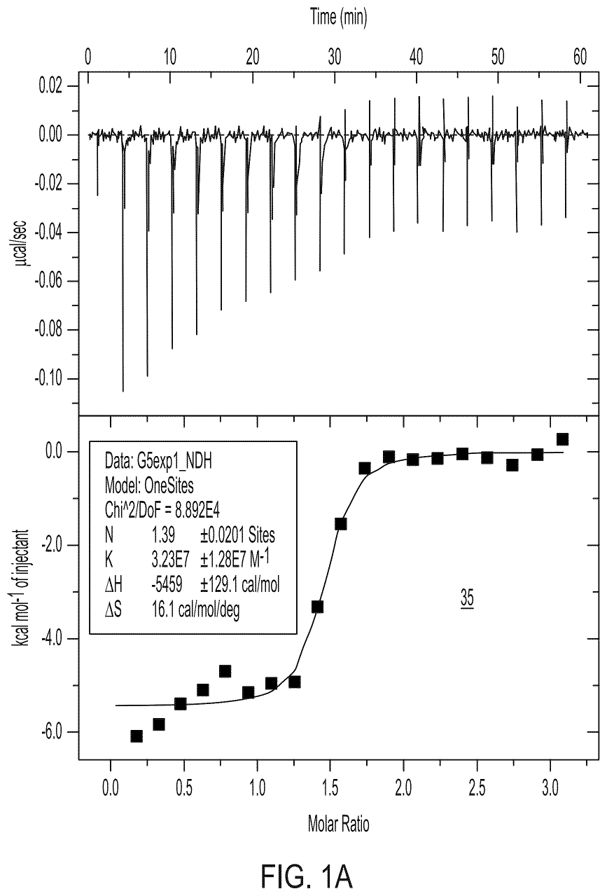

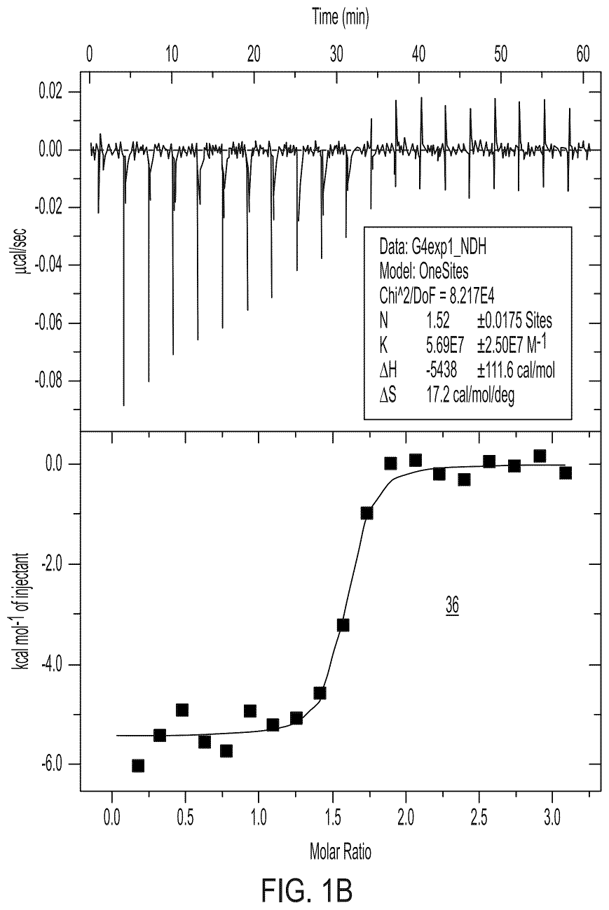

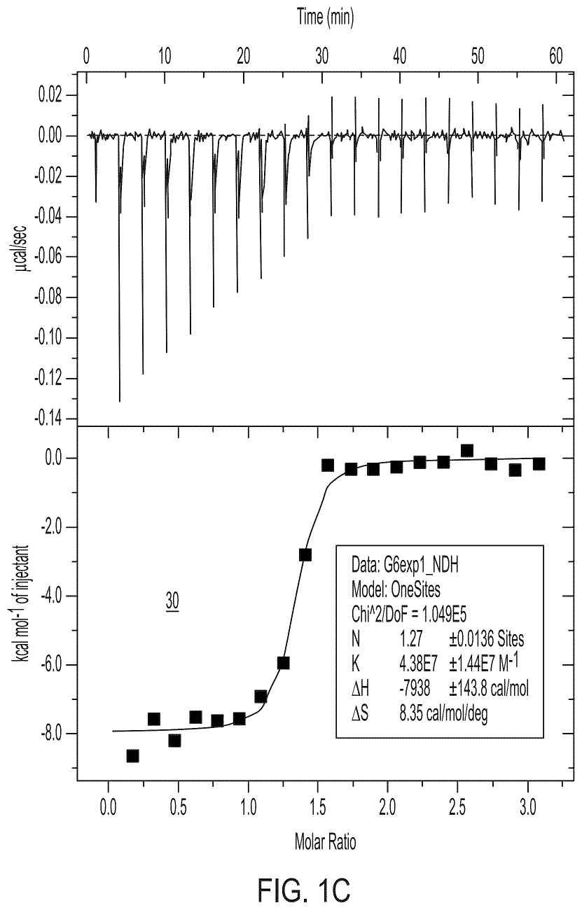

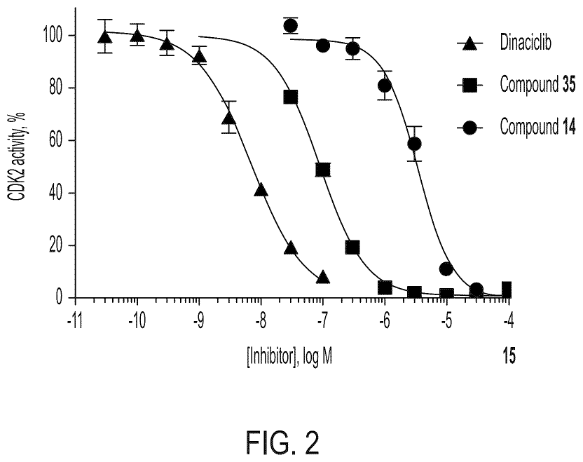

### DETAILED DESCRIPTION

The following definitions are used, unless otherwise described: halo or halogen is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkoxy” refers to an alkyl groups attached to the remainder of the molecule via an oxygen atom (“oxy”).

The term “alkylthio” refers to an alkyl groups attached to the remainder of the molecule via a sulfur atom.

The term “(C₃-C₆)carbocycle” refers to a saturated or partially unsaturated (non-aromatic) all carbon ring having 3 to 6 carbon atoms.

The term “alkoxycarbonyl” as used herein refers to a group (alkyl)-O—C(═O)—, wherein the term alkyl has the meaning defined herein.

The term “alkanoyl” as used herein refers to a group (alkyl)-C(═O)—, wherein the term alkyl has the meaning defined herein.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” as used herein includes mammals.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

The term “contraceptive” as used here in refers a method, pharmaceutical agent or compound or device used to prevent or reduce the likelihood of pregnancy.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of Formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, or tert-butyl; and (C₁-C₆)alkoxy can be methoxy, ethoxy, propoxy, or isopropoxy.

In one embodiment, the compound of Formula (I) is a compound of Formula (Ia):

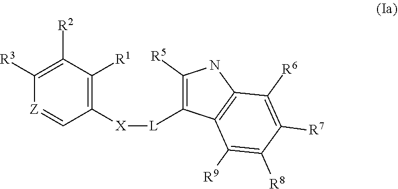

or a salt thereof, wherein:
R¹ is —COOR^{a} or tetrazol-5-yl;
R² is H, halo, amino, or hydroxy;
R³ is H, nitro, cyano, halo, —SCF₃, —SF₅, (C₁-C₄)alkyl, trifluoromethyl, or trifluoromethoxy; or R³ and R^{x} taken together with the atoms to which they are attached form a 6-membered carbocycle or phenyl ring;
R⁵ is H, (C₁-C₄)alkyl, or hydroxymethyl;
R⁶ is H, halo, or hydroxy;
R⁷ is H, halo, cyano, (C₁-C₄)alkyl, or (C₁-C₄)alkoxy, wherein any (C₁-C₄)alkyl and (C₁-C₄)alkoxy is optionally substituted with one or more fluoro;
R⁸ is H, halo, nitro, or cyano;
R⁹ is H, halo, or (C₁-C₄)alkyl;
Z is CR^{x} or N;
R^{a} is H or (C₁-C₆)alkyl;
R^{x} is H, halo, nitro, (C₁-C₄)alkyl, or (C₁-C₄)alkoxy; or R^{x} and R³ taken together with the atoms to which they are attached form a 6-membered carbocycle or phenyl ring;
X is S, O, or NH; and
L is absent or is (C₁-C₄)alkylene.

In one embodiment, R¹ is —COOR^{a}.

In one embodiment, R^{a} is H.

In one embodiment, R^{a} is (C₁-C₆)alkyl.

In one embodiment, R¹ is tetrazol-5-yl.

In one embodiment, R² is H.

In one embodiment, R² is amino or hydroxy.

In one embodiment, R³ is nitro.

In one embodiment, R³ is cyano.

In one embodiment, R³ is halo.

In one embodiment, R³ is —SF₅.

In one embodiment, R³ is trifluoromethyl.

In one embodiment, R³ is H.

In one embodiment, R³ is —SCF₃.

In one embodiment, R³ is trifluoromethoxy.

In one embodiment, R^{x} and R³ taken together with the atoms to which they are attached form a 6-membered carbocycle.

In one embodiment, R^{x} and R³ taken together with the atoms to which they are attached form a phenyl.

In one embodiment, R⁵ is H.

In one embodiment, R⁵ is hydroxymethyl.

In one embodiment, R⁶ is H.

In one embodiment, R⁶ is halo.

In one embodiment, R⁶ is hydroxy.

In one embodiment, R⁷ is H.

In one embodiment, R⁷ is halo.

In one embodiment, R⁷ is cyano.

In one embodiment, R⁷ is (C₁-C₄)alkyl that is optionally substituted with one or more fluoro.

In one embodiment, R⁷ (C₁-C₄)alkoxy that is optionally substituted with one or more fluoro.

In one embodiment, R⁷ is H, fluoro, chloro, bromo, methyl, methoxy, trifluoromethoxy, or cyano.

In one embodiment, R⁸ is H.

In one embodiment, R⁸ is halo.

In one embodiment, R⁸ is nitro.

In one embodiment, R⁸ is cyano.

In one embodiment, R⁸ is H, chloro, fluoro, nitro, or cyano.

In one embodiment, R⁹ is H.

In one embodiment, R⁹ is halo.

In one embodiment, R⁹ is (C₁-C₄)alkyl.

In one embodiment, R⁹ is H, methyl, chloro, or fluoro.

In one embodiment, Z is CR^{x}.

In one embodiment, R^{x} is H, halo, nitro, (C₁-C₄)alkyl, or (C₁-C₄)alkoxy.

In one embodiment, R^{x} is halo, nitro, (C₁-C₄)alkyl, or (C₁-C₄)alkoxy;

In one embodiment, Z is N.

In one embodiment, X is NH.

In one embodiment, X is S.

In one embodiment, L is absent.

In one embodiment, L is (C₂-C₄)alkylene.

In one embodiment, L is —CH₂CH₂—, or —CH(CH₃)CH₂—.

One embodiment provides a compound of Formula (I):

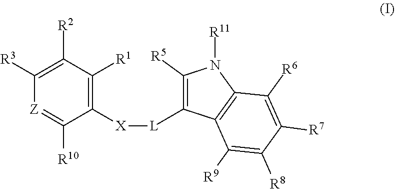

or a salt thereof, wherein:
R¹ is —COOR^{a} or tetrazol-5-yl;
R² is H, halo, nitro, trifluoromethyl, cyano, amino, or hydroxy;
R³ is H, nitro, cyano, halo, —SCF₃, —SF₅, (C₁-C₄)alkyl, (C₃-C₆)cycloalkyl, (C₁-C₄)alkoxycarbonyl, (C₁-C₄)alkanoyl, (C₁-C₄)alkoxy, phenyl, (C₁-C₄)alkylthio, trifluoromethyl, or trifluoromethoxy; or R³ and R^{x} taken together with the atoms to which they are attached form a 6-membered carbocycle or phenyl ring;
R⁵ is H, (C₁-C₄)alkyl, pyridyl, carboxy, or hydroxymethyl;
R⁶ is H, halo, (C₁-C₄)alkyl, or hydroxy;
R⁷ is H, halo, cyano, (C₁-C₄)alkyl, hydroxy, or (C₁-C₄)alkoxy, wherein any (C₁-C₄)alkyl and (C₁-C₄)alkoxy is optionally substituted with one or more fluoro;
R⁸ is H, halo, (C₁-C₄)alkyl, nitro, or cyano;
R⁹ is H, halo, hydroxy, or (C₁-C₄)alkyl;
R¹⁰ is H, halo, nitro, or (C₁-C₄)alkyl;
R¹¹ is H or (C₁-C₄)alkyl;
Z is CR^{x} or N;
R^{a} is H or (C₁-C₆)alkyl;
R^{x} is H, halo, nitro, (C₁-C₄)alkyl, amino, or (C₁-C₄)alkoxy; or R^{x} and R³ taken together with the atoms to which they are attached form a 5-membered carboocycle, 6-membered carbocycle, a 5-membered heterocycle, a 6-membered heterocycle, or phenyl ring;
X is S, O, or NH; and
L is absent or is (C₁-C₄)alkylene.

In one embodiment, the corn pound or salt is selected from the group consisting of:

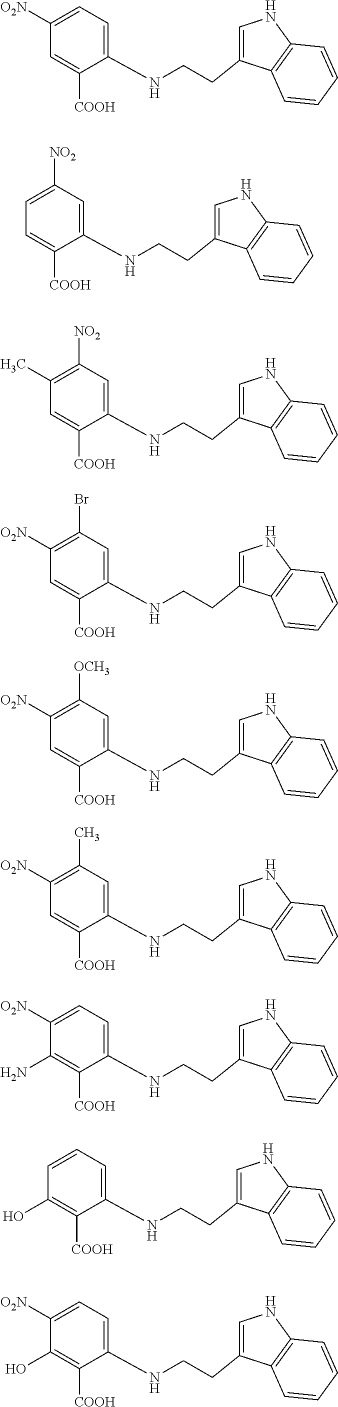

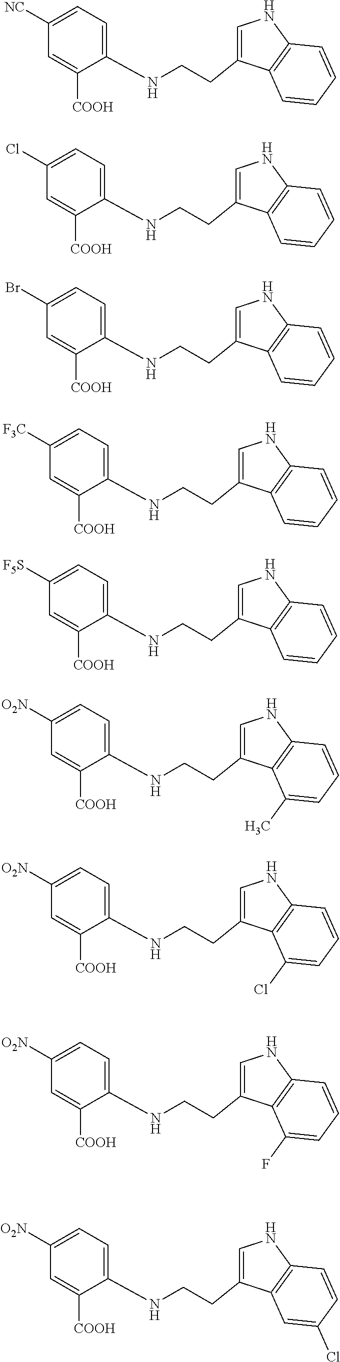

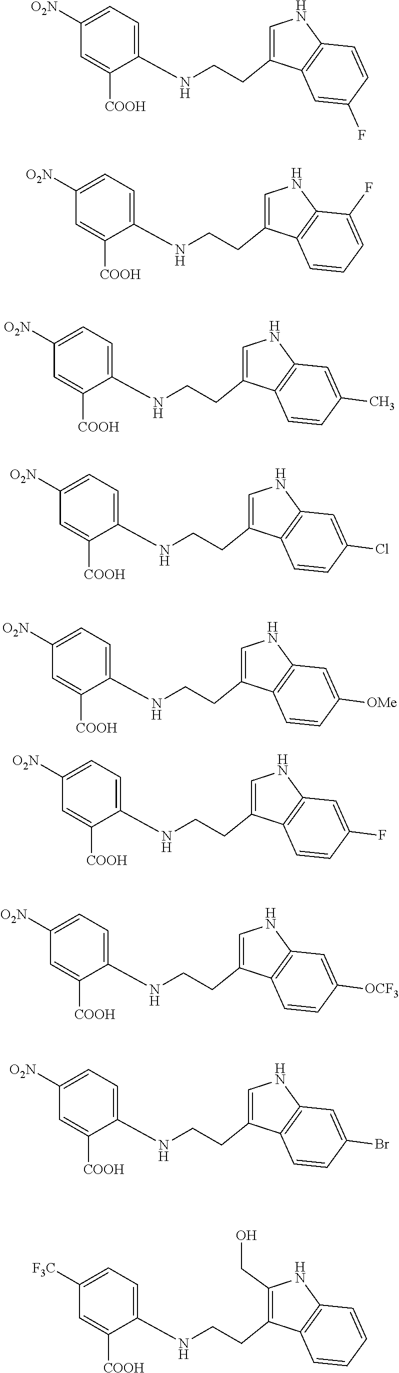

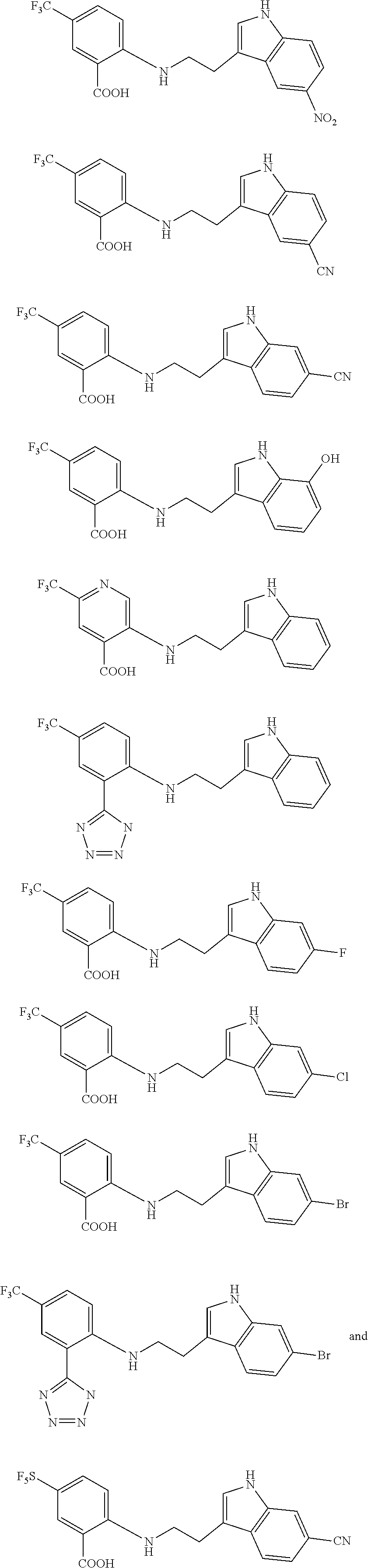

and salts thereof

In one embodiment, the compound or salt is selected from the group consisting of:

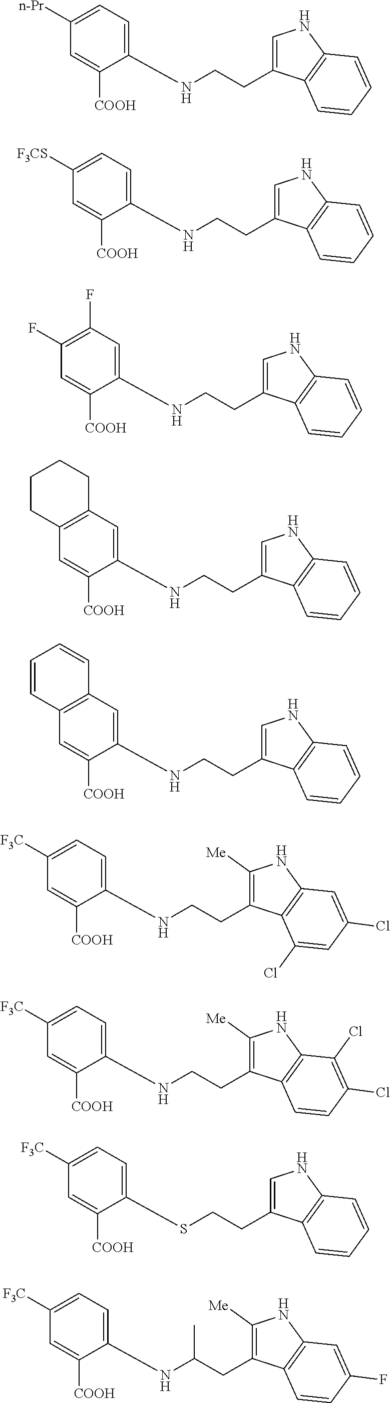

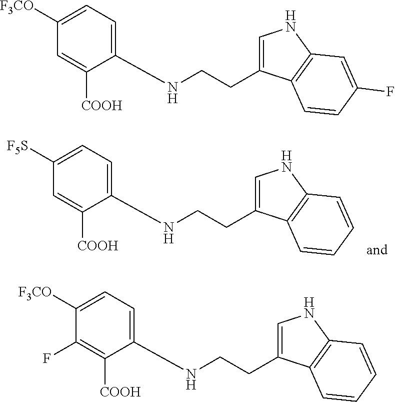

and salts thereof.

In one embodiment, when R^{x} and R³ taken together with the atoms to which they are attached form a 5-membered heterocycle or a 6-membered heterocycle, the group:

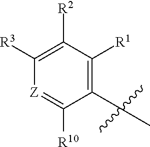

can be selected from:

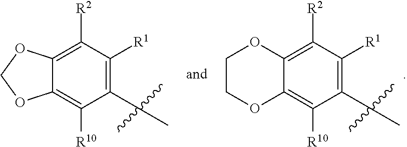

Processes for preparing compounds of Formula (I) are provided as further embodiments of the invention and are illustrated by the following procedures in which the meanings of the generic radicals are as given above unless otherwise qualified.

In cases where compounds are sufficiently basic or acidic, a salt of a compound of Formula (I) can be useful as an intermediate for isolating or purifying a compound of Formula (I). Additionally, administration of a compound of Formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium, or lithium) or alkaline earth metal (for example calcium) salts of carboxylic acids can also be made.

The compounds of Formula (I) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or using surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of Formula (I) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of Formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for treating cancer. Examples of such agents include ATP-site CDK2 inhibitors, other kinase inhibitors (including CDK4/6 inhibitors), and antimitotic agents such as paclitaxel, docetaxel, ixabepilone, altretamine, capecitabine, cyclophosphamide, etoposide, gemcitabine, ifosfamide, irinotecan and platinum agents.

Compounds of the invention can also be administered in combination with other therapeutic agents, for example, other CDK2 inhibitors, inhibitors of other kinases (e.g., TSSK and HIPK4), retinoic acid inhibitors, sAC inhibitors, CatSper inhibitors, Na,K-ATPase-alpha4 inhibitors, Slo3 inhibitors, anti-eppin antibodies, or hormonal contraceptives.

Accordingly, in one embodiment the invention also provides a composition comprising a compound of Formula (I), or a pharmaceutically acceptable salt thereof, at least one other therapeutic agent, and a pharmaceutically acceptable diluent or carrier. The invention also provides a kit comprising a compound of Formula (I), or a pharmaceutically acceptable salt thereof, at least one other therapeutic agent, packaging material, and instructions for administering the compound of Formula (I) or the pharmaceutically acceptable salt thereof and the other therapeutic agent or agents to an animal to treat cancer or to produce a contraceptive effect.

The invention will now be illustrated by the following non-limiting Examples.

### EXAMPLES

General Synthetic Method for Preparing Compounds Wherein R¹ is COOR^{a} and X is NH

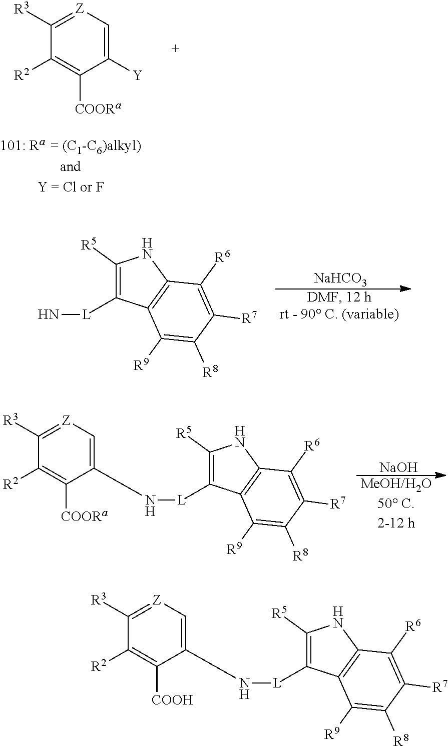

Alkyl 2-halobenzoic esters 101 (0.30 mmol) were dissolved in DMF (5 mL) with 1.1 equivalents of commercial tryptamine derivatives (in their neutral form or as HCl salts) and 3-4 equivalents of sodium bicarbonate. Potassium carbonate was often an acceptable substitute for NaHCO₃ and was sometimes used instead. The reaction temperature employed varied from room temperature to 90° C. and was dependent on the degree of electron-deficiency of the benzene ring and whether the substitution patterns around the benzene ring stabilize the Meisenheimer complex formed as a reactive intermediate in SₙAr. The solution was stirred for 12 hours. After, 1 M HCl (aq) was added until pH<7 and extracted two times into DCM. The DCM solvent was removed via rotary evaporation and the crude product was dissolved in MeOH to proceed to the hydrolysis step without purification. Water was added to the MeOH mixture until a 1:1 ratio of solvents was reached. A solution of 5 M NaOH (aq) (1 mL) was added, and the hydrolysis proceeded at 50° C. for 2-12 hours and monitored by TLC for completion. The mixture was cooled to room temperature and the solvent removed by rotary evaporation. 1 M HCl (aq) was added until pH<4 and the solution was extracted two times into EtOAc. The EtOAc layer was dried with MgSO₄ and loaded onto Celite. Subsequent purification was accomplished with silica gel flash column chromatography with a gradient of EtOAc in hexanes of 0-100% over 7 minutes, eluting around 60% EtOAc as the free carboxylic acid. The solvent was removed by rotary evaporation and isolated as the final product.

### General Synthetic Method for Preparing Examples 30 and 38

The synthesis of the nitrile tryptamine precursor used to make compounds 30 and 38 has been described previously (Chen, Z., et al., International Patent Application Publication Number WO 2002078693, 2002). One mmol 3-formyl-1H-indole-6-carbonitrile and two equivalents of ammonium acetate were refluxed in nitromethane (5 mL) for 1.5 hours, and then cooled to room temperature. The resulting nitroalkene was obtained via filtration, washed with water, and allowed to dry, resulting in a 91% yield.

The nitroalkene was dissolved in a 9:1 ratio of THF:MeOH (5 mL) under anhydrous conditions. NaBH₄ (1.3 equivalents) was added, and the mixture was allowed to stir at room temperature for 1.5 hours. Water (1 mL) was used to quench the reaction and the mixture was loaded onto Celite for flash column chromatography. The nitroalkane was purified by silica gel flash column chromatography with a gradient of EtOAc in hexanes of 0-100% in 7 minutes, eluting at 55% EtOAc with a 45% yield. Solvent was removed by rotary evaporation and the nitroalkane was dissolved in 1:1 ratio of MeOH:2 M HCl (aq) with 23 equivalents of Zn. The solution was refluxed for 2 h and the remaining Zn was filtered off. The solution was made basic (pH<13) with K₂CO₃ and loaded onto Celite. The tryptamine derivative 3-(2-aminoethyl)-1H-indole-6-carbonitrile was purified by silica gel flash column chromatography with a gradient of MeOH (0.1% triethylamine) in DCM (0.1% triethylamine) of 0-30% in 5 minutes, eluting at 23% MeOH as an oil. The product was combined with the respective methyl ester via the general SₙAr/hydrolysis method. The yield over the final three steps was 25% for Compound 6 and 24% for Compound 7.

### General Synthetic Method for Making Pentafluorosulfanyl (SF₅) Methyl Ester for Examples 14 and 38

Due to the lack of commercial availability of the methyl ester for SF₅ Example 14 and Example 38, the conversion of 2-fluoro-5-(pentafluoro-λ⁶-sulfaneyl)benzoic acid to its methyl ester was necessary. 2-Fluoro-5-(pentafluoro-λ⁶-sulfaneyl)benzoic acid (0.50 mmol) was added with 5 equivalents of methyl iodide in DMF (5 mL) under anhydrous conditions with 4 equivalents of K₂CO₃. The resulting mixture was stirred at 40° C. for 12 hours. The reaction was extracted into DCM two times and dried with MgSO₄, and then loaded onto Celite. Subsequent purification was accomplished by silica gel flash column chromatography with a gradient of EtOAc in hexanes of 0-100% over 7 minutes, eluting around 30% EtOAc as the ester in a 71% yield as an oil. The general SₙAr/hydrolysis method was employed to yield Example 14 and Example 38.

### General Synthesis Method for Making Tetrazoles 33 and 37.

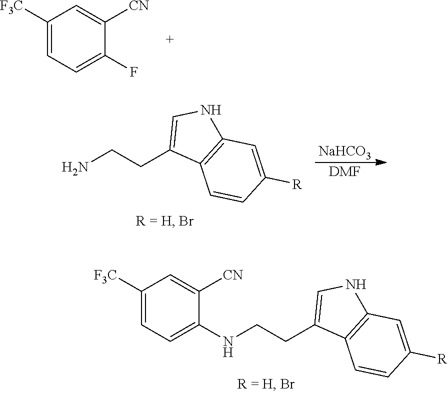

### Synthesis of Tetrazole Precursors 2-((2-(1H-Indol-3-yl)ethyl)amino)-5-(trifluoromethyl)benzonitrile and 2-((2-(6-Bromo-1H-indol-3-yl)ethyl)amino)-5-(trifluoromethyl)benzonitrile

Performed as described for the general method to yield carboxylic acid substitutions.

2-((2-(1H-Indol-3-yl)ethyl)amino)-5-(trifluoromethyl)benzonitrile: 91% yield. White solid. 1H NMR (400 MHz, DMSO-d₆) δ 10.88 (s, 1H), 7.88 (s, 1H), 7.69 (d, J=9.2 Hz, 1H), 7.59 (d, J=7.8 Hz, 1H), 7.36 (d, J=8.1 Hz, 1H), 7.23 (s, 1H), 7.13-7.04 (m, 1H), 7.04-6.95 (m, 2H), 6.90 (t, J=5.8 Hz, 1H), 3.55 (q, J=7.0 Hz, 2H), 3.00 (t, J=7.5 Hz, 2H). 19F NMR (376 MHz, DMSO) δ −59.7.

2-((2-(6-Bromo-1H-indol-3-yl)ethyl)amino)-5-(trifluoromethyl)benzonitrile: ¹H NMR (400 MHz, CDCl₃) δ 8.40 (s, 1H), 7.58 (s, 1H), 7.56-7.51 (m, 2H), 7.43 (d, J=8.4 Hz, 1H), 7.22 (dd, J=8.4, 1.7 Hz, 1H), 7.07 (s, 1H), 6.69 (d, J=8.9 Hz, 1H), 5.00 (t, J=5.4 Hz, 1H), 3.52 (q, J=6.5 Hz, 2H), 3.10 (t, J=6.7 Hz, 2H). ¹⁹F NMR (376 MHz, CDCl₃) δ −61.75.

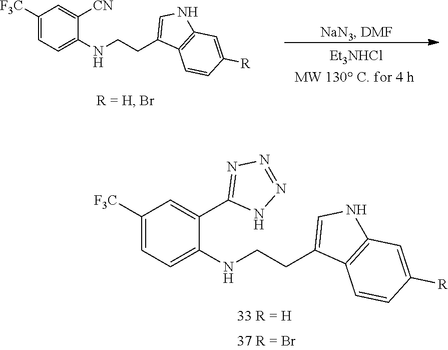

### Synthesis of Tetrazoles 33 and 37

Tetrazole synthesis started for 33 with nitrile precursor 2-((2-(1H-indol-3-yl)ethyl)amino)-5-(trifluoromethyl)benzonitrile and for 37 the analogous nitrile precursor 2-((2-(6-bromo-1H-indol-3-yl)ethyl)amino)-5-(trifluoromethyl)benzonitrile with a bromine substitution on the indole. The nitriles (0.50 mmol) were placed in a microwave vial and dissolved in DMF (5 mL) with 3 equivalents of both sodium azide and triethylammonium chloride. The vial was irradiated with microwaves at 130° C. for 4 h. The products 33 and 37 were extracted into EtOAc two times with HCl (aq), pH=4. The EtOAc layers were dried with MgSO4 and loaded onto Celite. Subsequent purification of 33 was accomplished with RediSep Rf Gold Silica Gel Disposable Flash columns from Teledyne Isco (4 g, 18 mL/min from rate) with a gradient of EtOAc in hexanes of 0-100% over 7 min, eluting at 45% EtOAc for 33 with a 68% yield. Purification of 37 was performed with a MeOH/DCM gradient of 0-30% MeOH in 5 min, eluting at 26% MeOH with a 7% yield.

### Example 1. Synthesis of

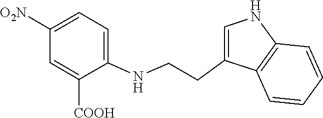 [SMILES: O=C(O)c1cc([N+](=O)[O-])ccc1NCCc1c[nH]c2ccccc12]

Yellow solid. m.p. 216-218° C. ¹H NMR (400 MHz, DMSO-d₆) δ 13.35 (s, 1H), 10.90 (s, 1H), 8.85 (s, 1H), 8.63 (s, 1H), 8.16 (d, J=9.3 Hz, 1H), 7.60 (d, J=7.8 Hz, 1H), 7.35 (d, J=8.1 Hz, 1H), 7.25 (s, 1H), 7.08 (t, J=7.5 Hz, 1H), 6.99 (t, J=7.4 Hz, 1H), 6.93 (d, J=9.6 Hz, 1H), 3.64 (q, J=6.9 Hz, 2H), 3.07 (t, J=7.0 Hz, 2H).

### Example 2. Synthesis of

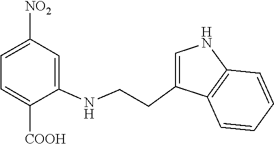 [SMILES: O=C(O)c1ccc([N+](=O)[O-])cc1NCCc1c[nH]c2ccccc12]

78% yield. Yellow solid. m.p. 216-218° C. ¹H NMR (400 MHz, DMSO-d₆) δ 13.35 (s, 1H), 10.90 (s, 1H), 8.85 (s, 1H), 8.63 (s, 1H), 8.16 (d, J=9.3 Hz, 1H), 7.60 (d, J=7.8 Hz, 1H), 7.35 (d, J=8.1 Hz, 1H), 7.25 (s, 1H), 7.08 (t, J=7.5 Hz, 1H), 6.99 (t, J=7.4 Hz, 1H), 6.93 (d, J=9.6 Hz, 1H), 3.64 (q, J=6.9 Hz, 2H), 3.07 (t, J=7.0 Hz, 2H).

### Example 3. Synthesis of

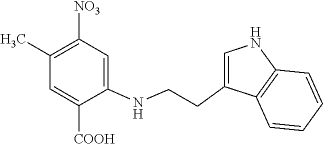

48% yield. Yellow solid. ¹H NMR (400 MHz, DMSO-d₆) δ 13.18 (s, 1H), 10.87 (s, 1H), 7.83 (s, 1H), 7.59 (d, J=7.9 Hz, 1H), 7.35 (d, J=8.1 Hz, 1H), 7.25 (s, 1H), 7.22 (s, 1H), 7.08 (t, J=7.6 Hz, 1H), 6.98 (t, J=7.4 Hz, 1H), 3.51 (t, J=7.0 Hz, 2H), 3.04 (t, J=6.9 Hz, 2H), 2.30 (s, 3H). ¹³C NMR (101 MHz, DMSO-d₆) δ 168.6, 153.0, 149.1, 136.3, 135.6, 133.6, 127.0, 123.1, 121.0, 118.3, 115.5, 113.4, 111.4, 111.2, 106.2, 42.9, 24.3, 17.6.

### Example 4. Synthesis of

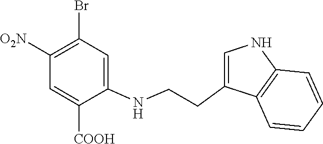 [SMILES: O=C(O)c1cc([N+](=O)[O-])c(Br)cc1NCCc1c[nH]c2ccccc12]

56% yield. Yellow solid. ¹H NMR (400 MHz, DMSO-d₆) δ 13.55 (s, 1H), 10.89 (s, 1H), 8.70 (s, 1H), 8.54 (s, 1H), 7.59 (d, J=7.9 Hz, 1H), 7.35 (d, J=8.1 Hz, 1H), 7.24 (s, 1H), 7.15 (s, 1H), 7.08 (t, J=7.5 Hz, 1H), 6.99 (t, J=7.5 Hz, 1H), 3.65 (q, J=6.4 Hz, 2H), 3.05 (t, J=6.9 Hz, 2H).

### Example 5. Synthesis of

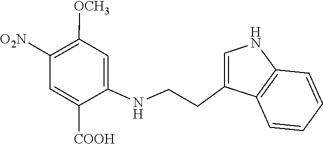 [SMILES: COc1cc(NCCc2c[nH]c3ccccc23)c(C(=O)O)cc1[N+](=O)[O-]]

21% yield. Yellow solid. ¹H NMR (400 MHz, DMSO-d₆) δ 13.07 (s, 1H), 10.87 (s, 1H), 8.76 (s, 1H), 8.51 (s, 1H), 7.60 (d, J=7.8 Hz, 1H), 7.34 (d, J=8.1 Hz, 1H), 7.22 (s, 1H), 7.07 (t, J=7.5 Hz, 1H), 6.98 (t, J=7.4 Hz, 1H), 6.21 (s, 1H), 3.84 (s, 3H), 3.64 (q, J=6.5 Hz, 2H), 3.07 (t, J=6.8 Hz, 2H).

### Example 6. Synthesis of

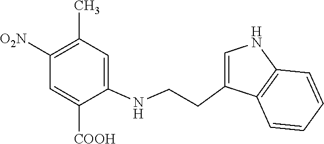 [SMILES: Cc1cc(NCCc2c[nH]c3ccccc23)c(C(=O)O)cc1[N+](=O)[O-]]

35% yield. Yellow solid. ¹H NMR (400 MHz, DMSO-d₆) δ 13.26 (s, 1H), 10.88 (s, 1H), 8.64-8.58 (m, 2H), 7.60 (d, J=7.9 Hz, 1H), 7.36 (d, J=8.0 Hz, 1H), 7.24 (s, 1H), 7.09 (t, J=7.5 Hz, 1H), 7.00 (t, J=7.4 Hz, 1H), 6.75 (s, 1H), 3.64 (q, J=6.2 Hz, 2H), 3.07 (t, J=6.9 Hz, 2H), 2.55 (s, 3H). ¹³C NMR (101 MHz, DMSO-d₆) δ 168.5, 153.3, 141.3, 136.3, 135.3, 130.5, 127.0, 123.3, 121.0, 118.32, 118.25, 114.0, 111.4, 110.9, 107.8, 42.9, 24.4, 21.9.

### Example 7. Synthesis of

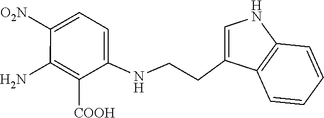 [SMILES: Nc1c([N+](=O)[O-])ccc(NCCc2c[nH]c3ccccc23)c1C(=O)O]

73% yield. Yellow solid. ¹H NMR (400 MHz, DMSO-d₆) δ 10.88 (s, 1H), 9.35-8.71 (m, 3H), 8.06 (d, J=9.9 Hz, 1H), 7.58 (d, J=7.9 Hz, 1H), 7.34 (d, J=8.1 Hz, 1H), 7.21 (s, 1H), 7.07 (t, J=7.5 Hz, 1H), 6.98 (t, J=7.4 Hz, 1H),

### Example 8. Synthesis of

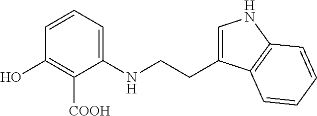 [SMILES: O=C(O)c1c(O)cccc1NCCc1c[nH]c2ccccc12]

1 5% yield. White solid. ¹H NMR (400 MHz, DMSO-d₆) δ 10.86 (s, 1H), 7.57 (d, J=7.3 Hz, 1H), 7.36 (s, 1H), 7.22-7.14 (m, 2H), 7.08 (t, J=7.4 Hz, 1H), 6.98 (t, J=7.6 Hz, 1H), 6.21 (d, J=8.2 Hz, 1H), 6.07 (d, J=7.9 Hz, 1H), 3.42 (t, J=7.0 Hz, 2H), 3.02 (t, J=6.9 Hz, 2H).

### Example 9. Synthesis of

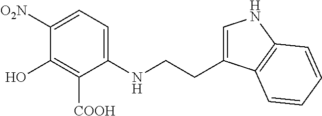 [SMILES: O=C(O)c1c(NCCc2c[nH]c3ccccc23)ccc([N+](=O)[O-])c1O]

72% yield. Yellow solid. ¹H NMR (400 MHz, DMSO-d₆) δ 10.88 (s, 1H), 10.12 (t, J=5.3 Hz, 1H), 8.39 (s, 1H), 7.85 (d, J=9.6 Hz, 1H), 7.58 (d, J=7.8 Hz, 1H), 7.35 (d, J=8.0 Hz, 1H), 7.22 (s, 1H), 7.07 (t, J=8.2 Hz, 1H), 6.98 (t, J=8.0 Hz, 1H), 6.02 (d, J=9.7 Hz, 1H), 3.50 (q, J=5.3 Hz, 2H), 3.00 (t, J=7.1 Hz, 2H).

### Example 10. Synthesis of

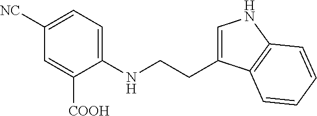 [SMILES: N#Cc1ccc(NCCc2c[nH]c3ccccc23)c(C(=O)O)c1]

24% yield over two steps. Light yellow solid. m.p. 247° C. ¹H NMR (400 MHz, DMSO-d₆) δ 13.12 (s, 1H), 10.87 (s, 1H), 8.53 (s, 1H), 8.07 (s, 1H), 7.68 (d, J=11.1 Hz, 1H), 7.58 (d, J=7.8 Hz, 1H), 7.34 (d, J=8.1 Hz, 1H), 7.23 (d, J=2.3 Hz, 1H), 7.07 (t, J=8.2 Hz, 1H), 6.98 (t, J=7.5 Hz, 1H), 6.91 (d, J=9.0 Hz, 1H), 3.63-3.53 (m, 2H), 3.04 (t, J=7.0 Hz, 2H). ¹³C NMR (101 MHz, DMSO-d₆) δ 168.5, 152.8, 136.9, 136.5, 136.3, 127.0, 123.2, 121.0, 119.6, 118.30, 118.28, 112.3, 111.4, 111.0, 110.2, 95.0, 42.6, 24.3.

### Example 11. Synthesis of

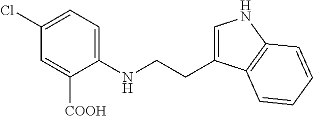 [SMILES: O=C(O)c1cc(Cl)ccc1NCCc1c[nH]c2ccccc12]

19% yield. White solid. ¹H NMR (400 MHz, DMSO-d₆) δ 10.86 (s, 1H), 7.70 (s, 1H), 7.58 (d, J=7.8 Hz, 1H), 7.41-7.31 (m, 2H), 7.22 (s, 1H), 7.07 (t, J=7.5 Hz, 1H), 6.98 (t, J=7.4 Hz, 1H), 6.83 (d, J=9.1 Hz, 1H), 3.47 (t, J=7.0 Hz, 2H), 3.02 (t, J=6.9 Hz, 2H).

### Example 12. Synthesis of

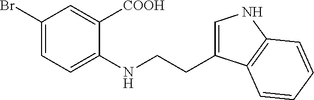 [SMILES: O=C(O)c1cc(Br)ccc1NCCc1c[nH]c2ccccc12]

27% yield over two steps. White solid. ¹H NMR (400 MHz, DMSO-d₆) δ 10.86 (s, 1H), 7.83 (d, J=2.5 Hz, 1H), 7.57 (d, J=7.8 Hz, 1H), 7.48 (dd, J=9.0, 2.6 Hz, 1H), 7.34 (d, J=8.1 Hz, 1H), 7.21 (s, 1H), 7.07 (t, J=6.9 Hz, 1H), 6.98 (t, J=8.0 Hz, 1H), 6.78 (d, J=9.1 Hz, 1H), 3.47 (t, J=7.0 Hz, 2H), 3.02 (t, J=6.9 Hz, 2H).

### Example 13. Synthesis of

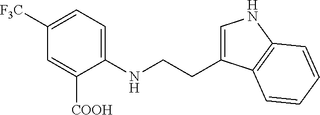 [SMILES: O=C(O)c1cc(C(F)(F)F)ccc1NCCc1c[nH]c2ccccc12]

33% yield. Light yellow solid. m.p. 201-202° C. ¹H NMR (400 MHz, DMSO-d₆) δ 13.05 (s, 1H), 10.88 (s, 1H), 8.34 (s, 1H), 8.02 (s, 1H), 7.63 (d, J=9.0 Hz, 1H), 7.59 (d, J=7.9 Hz, 1H), 7.35 (d, J=8.1 Hz, 1H), 7.23 (s, 1H), 7.08 (t, J=8.2 Hz, 1H), 7.01-6.92 (m, 2H), 3.55 (t, J=7.2 Hz, 2H), 3.05 (t, J=7.0 Hz, 2H).

### Example 14. Synthesis of

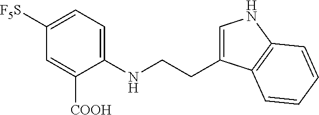

40% yield over three steps. Colorless solid. m.p. 237-238° C. ¹H NMR (400 MHz, DMSO-d₆) δ 13.31 (s, 1H), 10.89 (s, 1H), 8.40 (s, 1H), 8.15 (d, J=2.9 Hz, 1H), 7.80 (dd, J=9.5, 2.9 Hz, 1H), 7.59 (d, J=7.8 Hz, 1H), 7.35 (d, J=8.1 Hz, 1H), 7.24 (s, 1H), 7.08 (t, J=6.9 Hz, 1H), 6.98 (t, J=8.0 Hz, 1H), 6.92 (d, J=9.5 Hz, 1H), 3.56 (t, J=7.1 Hz, 2H), 3.05 (t, J=7.0 Hz, 2H).

### Example 15. Synthesis of

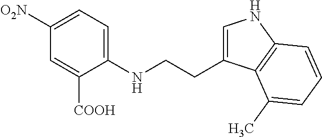 [SMILES: Cc1cccc2[nH]cc(CCNc3ccc([N+](=O)[O-])cc3C(=O)O)c12]

93% yield. Yellow solid. ¹H NMR (400 MHz, DMSO-d₆) δ 13.45 (s, 1H), 10.91-10.86 (m, 1H), 8.90 (s, 1H), 8.64 (d, J=2.8 Hz, 1H), 8.17 (dd, J=9.5, 2.9 Hz, 1H), 7.21-7.14 (m, 2H), 7.05-6.89 (m, 2H), 6.72 (d, J=7.0 Hz, 1H), 3.65 (q, J=6.6 Hz, 2H), 3.23 (t, J=6.9 Hz, 2H), 2.65 (s, 3H), 2.62 (s, 2H). ¹³C NMR (101 MHz, DMSO-d₆) δ 168.6, 154.5, 136.7, 134.5, 129.4, 128.6, 125.4, 123.4, 121.0, 120.0, 111.8, 111.6, 109.4, 44.1, 26.0, 20.1.

### Example 16. Synthesis of

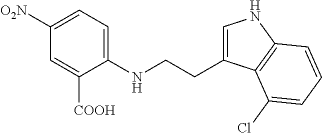 [SMILES: O=C(O)c1cc([N+](=O)[O-])ccc1NCCc1c[nH]c2cccc(Cl)c12]

68% yield. Yellow solid. ¹H NMR (400 MHz, DMSO-d₆) δ 11.29 (s, 1H), 8.67 (s, 1H), 8.00 (dd, J=9.2, 2.6 Hz, 1H), 7.37-7.31 (m, 2H), 7.09-6.98 (m, 2H), 6.72 (d, J=9.5 Hz, 1H), 3.57 (q, J=6.6 Hz, 2H), 3.25 (t, J=7.2 Hz, 2H).

### Example 17. Synthesis of

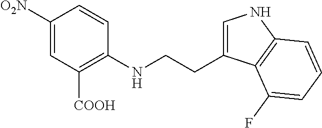 [SMILES: O=C(O)c1cc([N+](=O)[O-])ccc1NCCc1c[nH]c2cccc(F)c12]

45% yield. Yellow solid. ¹H NMR (400 MHz, DMSO-d₆) δ 11.24 (s, 1H), 10.03 (s, 1H), 8.66 (d, J=2.9 Hz, 1H), 8.06 (dd, J=9.3, 2.9 Hz, 1H), 7.26 (s, 1H), 7.19 (d, J=8.1 Hz, 1H), 7.03 (td, J=7.9, 5.2 Hz, 1H), 6.80 (d, J=9.4 Hz, 1H), 6.74 (dd, J=11.6, 7.8 Hz, 1H), 3.57 (q, J=6.6 Hz, 2H), 3.11 (t, J=7.1 Hz, 2H). ¹⁹F NMR (376 MHz, DMSO-d₆) δ −124.9. ¹³C NMR (101 MHz, DMSO-d₆) δ 169.0, 157.6, 155.2, 154.9, 139.4, 139.2, 134.1, 128.4, 127.9, 124.0, 121.5, 121.5, 115.3, 115.1, 110.1, 109.39, 109.36, 108.11, 108.08, 103.4, 103.2, 43.5, 25.7.

### Example 18. Synthesis of

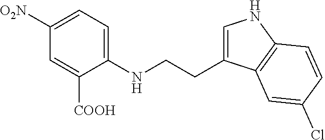 [SMILES: O=C(O)c1cc([N+](=O)[O-])ccc1NCCc1c[nH]c2ccc(Cl)cc12]

37% yield. Yellow solid. ¹H NMR (400 MHz, DMSO-d₆) δ 13.42 (s, 1H), 11.10 (s, 1H), 8.88 (s, 1H), 8.63 (d, J=2.9 Hz, 1H), 8.16 (dd, J=9.5, 2.9 Hz, 1H), 7.66 (s, 1H), 7.36 (d, J=8.6 Hz, 1H), 7.34 (s, 1H), 7.07 (dd, J=8.6, 2.1 Hz, 1H), 6.96 (d, J=9.6 Hz, 1H), 3.66-3.60 (m, 2H), 3.04 (t, J=7.1 Hz, 2H).

### Example 19. Synthesis of

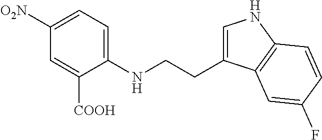 [SMILES: O=C(O)c1cc([N+](=O)[O-])ccc1NCCc1c[nH]c2ccc(F)cc12]

16% yield. Yellow solid. ¹H NMR (400 MHz, DMSO-d₆) δ 13.43 (s, 1H), 10.99 (s, 1H), 8.88 (s, 1H), 8.62 (s, 1H), 8.17 (dd, J=9.5, 2.7 Hz, 1H), 7.42-7.29 (m, 3H), 6.99-6.86 (m, 2H), 3.68-3.58 (m, 2H), 3.03 (t, J=7.0 Hz, 2H). ¹⁹F NMR (376 MHz, DMSO-d₆) δ −125.5 (td, J=9.7, 4.5 Hz). ¹³C NMR (101 MHz, DMSO-d₆) δ 168.6, 157.8, 155.5, 154.6, 134.5, 132.9, 129.3, 128.6, 127.3, 127.2, 125.4, 112.3, 112.2, 111.7, 111.3, 111.2, 109.2, 109.0, 103.3, 103.0, 42.9, 24.2.

### Example 20. Synthesis of

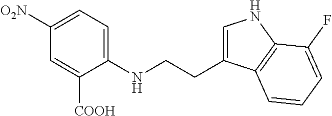 [SMILES: O=C(O)c1cc([N+](=O)[O-])ccc1NCCc1c[nH]c2c(F)cccc12]

17% yield. Yellow solid. ¹H NMR (400 MHz, DMSO-d₆) δ 13.44 (s, 1H), 11.40 (s, 1H), 8.88 (s, 1H), 8.63 (d, J=2.8 Hz, 1H), 8.17 (dd, J=9.5, 2.9 Hz, 1H), 7.43 (d, J=7.6 Hz, 1H), 7.33 (s, 1H), 6.98-6.86 (m, 3H), 3.70-3.61 (m, 2H), 3.07 (t, J=7.0 Hz, 2H). ¹⁹F NMR (376 MHz, DMSO-d₆) δ −133.8 (dd, J=11.5, 5.2 Hz). ¹³C NMR (101 MHz, DMSO-d₆) δ 168.6, 154.5, 148.0, 134.5, 131.1, 129.4, 128.6, 124.5, 124.0, 123.9, 118.7, 118.6, 114.6, 112.1, 111.6, 105.9, 105.8, 42.9, 24.2.

### Example 21. Synthesis of

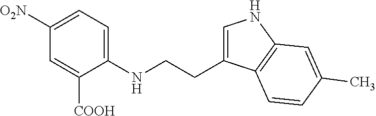 [SMILES: Cc1ccc2c(CCNc3ccc([N+](=O)[O-])cc3C(=O)O)c[nH]c2c1]

48% yield. Yellow solid. ¹H NMR (400 MHz, DMSO-d₆) δ 13.42 (s, 1H), 10.72 (s, 1H), 8.85 (s, 1H), 8.63 (d, J=2.9 Hz, 1H), 8.18 (dd, J=9.4, 2.9 Hz, 1H), 7.47 (d, J=8.1 Hz, 1H), 7.17-7.11 (m, 2H), 6.95 (d, J=9.5 Hz, 1H), 6.82 (dd, J=8.1, 1.5 Hz, 1H), 3.63 (q, J=6.7 Hz, 2H), 3.03 (t, J=7.0 Hz, 2H), 2.38 (s, 3H).

### Example 22. Synthesis of

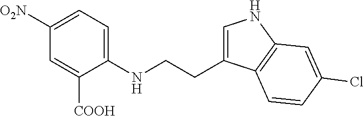 [SMILES: O=C(O)c1cc([N+](=O)[O-])ccc1NCCc1c[nH]c2cc(Cl)ccc12]

31% yield. Yellow solid. ¹H NMR (400 MHz, DMSO-d₆) δ 13.43 (s, 1H), 11.05 (s, 1H), 8.85 (s, 1H), 8.62 (s, 1H), 8.17 (d, J=7.3 Hz, 1H), 7.61 (d, J=8.6 Hz, 1H), 7.39 (s, 1H), 7.30 (s, 1H), 7.03-6.92 (m, 2H), 3.64 (q, J=6.6 Hz, 2H), 3.05 (t, J=7.1 Hz, 2H). ¹³C NMR (101 MHz, DMSO-d₆) δ 168.6, 154.5, 136.6, 134.5, 129.4, 128.6, 125.9, 125.8, 124.5, 119.8, 118.7, 111.7, 111.3, 111.0, 109.1, 42.9, 24.1.

### Example 23. Synthesis of

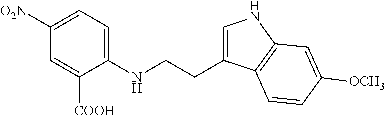 [SMILES: COc1ccc2c(CCNc3ccc([N+](=O)[O-])cc3C(=O)O)c[nH]c2c1]

Yellow solid. ¹H NMR (400 MHz, DMSO-d₆) δ 13.37 (s, 1H), 10.68 (s, 1H), 8.86 (s, 1H), 8.63 (s, 1H), 8.16 (d, J=9.6 Hz, 1H), 7.46 (d, J=8.7 Hz, 1H), 7.09 (s, 1H), 6.93 (d, J=9.6 Hz, 1H), 6.85 (s, 1H), 6.64 (d, J=8.7 Hz, 1H), 3.75 (s, 3H), 3.61 (t, J=6.4 Hz, 2H), 3.02 (t, J=6.6 Hz, 2H).

### Example 24. Synthesis of

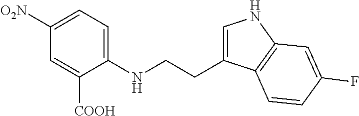 [SMILES: O=C(O)c1cc([N+](=O)[O-])ccc1NCCc1c[nH]c2cc(F)ccc12]

42% yield. Yellow solid. ¹H NMR (400 MHz, DMSO-d₆) δ 10.96 (s, 1H), 8.85 (t, J=5.2 Hz, 1H), 8.62 (d, J=2.9 Hz, 1H), 8.16 (dd, J=9.5, 3.0 Hz, 1H), 7.58 (dd, J=8.8, 5.4 Hz, 1H), 7.24 (s, 1H), 7.11 (dd, J=10.1, 2.4 Hz, 1H), 6.94 (d, J=9.5 Hz, 1H), 6.83 (td, J=9.2, 2.4 Hz, 1H), 3.63 (q, J=6.6 Hz, 2H), 3.04 (t, J=7.0 Hz, 2H). ¹⁹F NMR (376 MHz, DMSO-d₆) δ −122.3 (td, J=9.7, 5.5 Hz).

### Example 25. Synthesis of

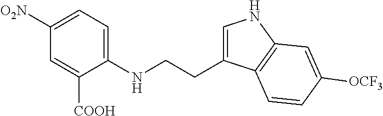 [SMILES: O=C(O)c1cc([N+](=O)[O-])ccc1NCCc1c[nH]c2cc(OC(F)(F)F)ccc12]

34% yield. Yellow solid. ¹H NMR (400 MHz, DMSO-d₆) δ 13.42 (s, 1H), 11.14 (s, 1H), 8.86 (s, 1H), 8.62 (d, J=2.8 Hz, 1H), 8.16 (dd, J=9.5, 2.9 Hz, 1H), 7.69 (d, J=8.6 Hz, 1H), 7.36 (s, 1H), 7.31 (s, 1H), 6.99-6.92 (m, 2H), 3.64 (q, J=6.7 Hz, 2H), 3.07 (t, J=7.0 Hz, 2H). ¹⁹F NMR (376 MHz, DMSO-d₆) δ −56.8.

### Example 26. Synthesis of

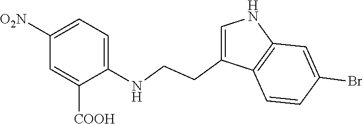 [SMILES: O=C(O)c1cc([N+](=O)[O-])ccc1NCCc1c[nH]c2cc(Br)ccc12]

91% yield. Yellow solid. ¹H NMR (400 MHz, DMSO-d₆) δ 13.43 (s, 1H), 11.05 (s, 1H), 8.85 (s, 1H), 8.63 (d, J=2.8 Hz, 1H), 8.17 (dd, J=9.4, 2.9 Hz, 1H), 7.57 (d, J=8.5 Hz, 1H), 7.53 (s, 1H), 7.33-7.26 (m, 1H), 7.11 (dd, J=8.4, 1.8 Hz, 1H), 6.96 (d, J=9.6 Hz, 1H), 3.64 (q, J=6.6 Hz, 2H), 3.05 (t, J=7.0 Hz, 2H).

### Example 27. Synthesis of

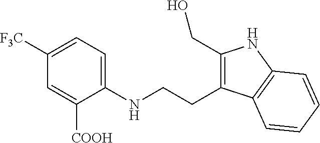 [SMILES: O=C(O)c1cc(C(F)(F)F)ccc1NCCc1c(CO)[nH]c2ccccc12]

White solid. ¹H NMR (400 MHz, DMSO-d₆) δ 10.89 (s, 1H), 8.36 (s, 1H), 8.01 (s, 1H), 7.62 (d, J=8.9 Hz, 1H), 7.54 (d, J=7.8 Hz, 1H), 7.30 (d, J=8.0 Hz, 1H), 7.04 (t, J=7.5 Hz, 1H), 6.99-6.90 (m, 2H), 4.61 (s, 2H), 3.50-3.44 (m, 2H), 3.04 (t, J=7.0 Hz, 2H). ¹⁹F NMR (376 MHz, DMSO-d₆) δ −59.5.

### Example 28. Synthesis of

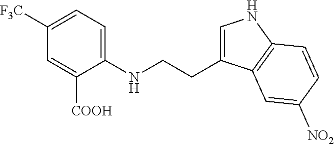 [SMILES: O=C(O)c1cc(C(F)(F)F)ccc1NCCc1c[nH]c2ccc([N+](=O)[O-])cc12]

39% yield. Yellow solid. ¹H NMR (400 MHz, DMSO-d₆) δ 13.07 (s, 1H), 11.67 (s, 1H), 8.59 (d, J=2.3 Hz, 1H), 8.35 (s, 1H), 8.04-7.95 (m, 2H), 7.63 (dd, J=9.0, 2.4 Hz, 1H), 7.55-7.48 (m, 2H), 6.98 (d, J=9.0 Hz, 1H), 3.59 (t, J=7.0 Hz, 2H), 3.13 (t, J=6.9 Hz, 2H). ¹⁹F NMR (376 MHz, DMSO-d₆) δ −59.6.

### Example 29. Synthesis of

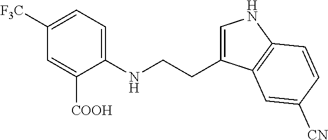 [SMILES: N#Cc1ccc2[nH]cc(CCNc3ccc(C(F)(F)F)cc3C(=O)O)c2c1]

70% yield. White solid. ¹H NMR (400 MHz, DMSO-d₆) δ 13.09 (s, 1H), 11.49 (s, 1H), 8.34 (s, 1H), 8.18 (s, 1H), 8.02 (s, 1H), 7.63 (dd, J=8.9, 2.4 Hz, 1H), 7.51 (d, J=8.4 Hz, 1H), 7.46 (s, 1H), 7.42 (dd, J=8.4, 1.6 Hz, 1H), 6.98 (d, J=9.0 Hz, 1H), 3.57 (t, J=6.9 Hz, 2H), 3.08 (t, J=7.0 Hz, 2H). ¹⁹F NMR (376 MHz, DMSO-d₆) δ −59.4. ¹³C NMR (101 MHz, DMSO-d₆) δ 168.9, 152.7, 137.9, 130.6, 130.6, 128.8, 127.0, 126.1, 125.9, 124.4, 123.7, 123.4, 120.9, 114.4, 114.1, 113.7, 113.4, 112.7, 112.6, 112.0, 109.2, 100.4, 42.7, 24.0.

### Example 30. Synthesis of

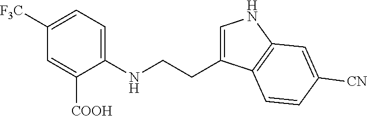 [SMILES: N#Cc1ccc2c(CCNc3ccc(C(F)(F)F)cc3C(=O)O)c[nH]c2c1]

9% yield over 5 steps. Colorless solid. m.p. 190-191° C. ¹H NMR (400 MHz, DMSO-d₆) δ 13.13-13.08 (m, 1H), 11.50 (s, 1H), 8.37 (s, 1H), 8.01 (s, 1H), 7.85 (s, 1H), 7.78 (d, J=8.2 Hz, 1H), 7.63 (dd, J=8.9, 2.4 Hz, 1H), 7.57 (s, 1H), 7.32 (dd, J=8.2, 1.5 Hz, 1H), 6.96 (d, J=9.0 Hz, 1H), 3.56 (t, J=7.0 Hz, 2H), 3.07 (t, J=7.0 Hz, 2H).

### Example 31. Synthesis of

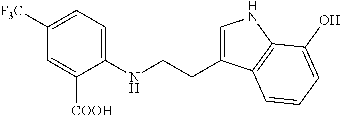 [SMILES: O=C(O)c1cc(C(F)(F)F)ccc1NCCc1c[nH]c2c(O)cccc12]

22% yield over 3 steps. White solid. ¹H NMR (400 MHz, DMSO-d₆) δ 13.10 (s, 1H), 10.67 (s, 1H), 9.45 (s, 1H), 8.33 (s, 1H), 8.01 (s, 1H), 7.64 (dd, J=9.0, 2.4 Hz, 1H), 7.12 (s, 1H), 7.03 (d, J=7.9 Hz, 1H), 6.95 (d, J=9.0 Hz, 1H), 6.78 (t, J=7.7 Hz, 1H), 6.49 (d, J=7.4 Hz, 1H), 3.53 (t, J=7.0 Hz, 2H), 3.00 (t, J=7.0 Hz, 2H). ¹⁹F NMR (376 MHz, DMSO-d₆) δ −59.6. ¹³C NMR (101 MHz, DMSO-d₆) δ 168.9, 152.7, 143.5, 130.7, 129.0, 128.8, 126.3, 126.1, 123.4, 122.6, 119.0, 114.0, 113.7, 111.9, 111.4, 109.4, 109.2, 105.4, 42.7, 24.5.

### Example 32. Synthesis of

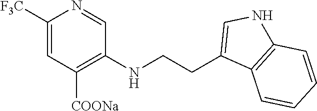

97% yield. White solid. ¹H NMR (400 MHz, DMSO-d₆) δ 10.85 (s, 1H), 9.80 (t, J=5.5 Hz, 1H), 8.04 (s, 1H), 7.92 (s, 1H), 7.58 (d, J=7.9 Hz, 1H), 7.34 (d, J=8.0 Hz, 1H), 7.23 (s, 1H), 7.06 (t, J=6.9 Hz, 1H), 6.98 (t, J=6.8 Hz, 1H), 3.50 (q, J=6.7 Hz, 2H), 3.01 (t, J=7.3 Hz, 2H). ¹⁹F NMR (376 MHz, DMSO-d₆) δ −64.0.

### Example 33. Synthesis of

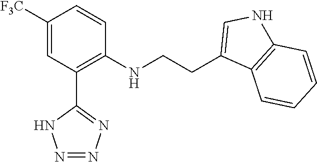 [SMILES: FC(F)(F)c1ccc(NCCc2c[nH]c3ccccc23)c(-c2nnn[nH]2)c1]

68% yield. White solid. ¹H NMR (400 MHz, DMSO-d₆) δ 10.89 (s, 1H), 8.20 (s, 1H), 7.66 (d, J=8.9 Hz, 1H), 7.60 (d, J=7.8 Hz, 1H), 7.34 (d, J=8.1 Hz, 1H), 7.28 (s, 1H), 7.12-7.03 (m, 2H), 6.97 (t, J=7.4 Hz, 1H), 3.65 (t, J=7.1 Hz, 2H), 3.35 (s, 1H), 3.12 (t, J=7.0 Hz, 2H).

### Example 34. Synthesis of

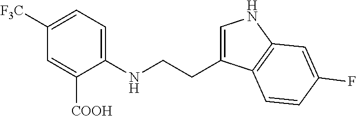 [SMILES: O=C(O)c1cc(C(F)(F)F)ccc1NCCc1c[nH]c2cc(F)ccc12]

84% yield. White solid. 1H NMR (400 MHz, DMSO-d₆) δ 13.10 (s, 1H), 10.96 (s, 1H), 8.37 (s, 1H), 8.01 (s, 1H), 7.63 (dd, J=8.9, 2.4 Hz, 1H), 7.58 (dd, J=8.7, 5.4 Hz, 1H), 7.24 (s, 1H), 7.12 (dd, J=10.2, 2.4 Hz, 1H), 6.95 (d, J=9.0 Hz, 1H), 6.89-6.79 (m, 1H), 3.55 (t, J=6.9 Hz, 2H), 3.03 (t, J=7.0 Hz, 2H). 19F NMR (376 MHz, DMSO-d₆) δ −59.6, −122.3. 13C NMR (101 MHz, DMSO) δ 157.7, 152.7, 136.0, 130.6, 128.8, 126.1, 123.9, 123.7, 119.4, 119.3, 111.8, 111.5, 106.9, 106.6, 97.4, 97.2, 42.7, 24.3.

### Example 35. Synthesis of

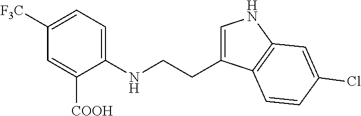 [SMILES: O=C(O)c1cc(C(F)(F)F)ccc1NCCc1c[nH]c2cc(Cl)ccc12]

70% yield. Colorless solid. m.p. 207-210° C. ¹H NMR (400 MHz, DMSO-d₆) δ 13.09 (s, 1H), 11.03 (s, 1H), 8.33 (s, 1H), 8.01 (s, 1H), 7.67-7.57 (m, 2H), 7.39 (s, 1H), 7.29 (s, 1H), 6.99 (dd, J=8.4, 1.9 Hz, 1H), 6.96 (d, J=9.0 Hz, 1H), 3.57-3.53 (m, 2H), 3.03 (t, J=7.0 Hz, 2H).

### Example 36. Synthesis of

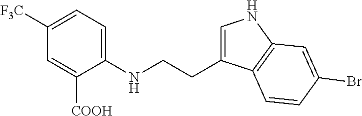 [SMILES: O=C(O)c1cc(C(F)(F)F)ccc1NCCc1c[nH]c2cc(Br)ccc12]

44% yield. Colorless solid. m.p. 213-214° C. ¹H NMR (400 MHz, DMSO-d₆) δ 13.10 (s, 1H), 11.04 (s, 1H), 8.33 (s, 1H), 8.01 (s, 1H), 7.63 (d, J=8.9 Hz, 1H), 7.60-7.51 (m, 1H), 7.28 (s, 1H), 7.11 (d, J=8.4 Hz, 1H), 6.96 (d, J=9.0 Hz, 1H), 3.57-3.52 (m, 2H), 3.03 (t, J=7.1 Hz, 2H).

### Example 37. Synthesis of

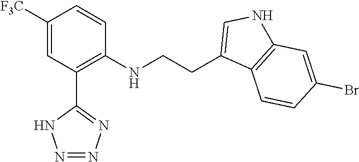 [SMILES: FC(F)(F)c1ccc(NCCc2c[nH]c3cc(Br)ccc23)c(-c2nnn[nH]2)c1]

7% yield over two steps. White solid. ¹H NMR (400 MHz, DMSO-d₆) δ 11.04 (s, 1H), 8.21 (s, 1H), 7.64 (d, J=9.4 Hz, 1H), 7.58 (d, J=8.4 Hz, 1H), 7.52 (s, 1H), 7.32 (s, 1H), 7.13-7.03 (m, 2H), 3.63 (t, J=7.2 Hz, 2H), 3.10 (t, J=7.0 Hz, 2H). ¹⁹F NMR (376 MHz, DMSO-d₆) δ −59.2.

### Example 38. Synthesis of

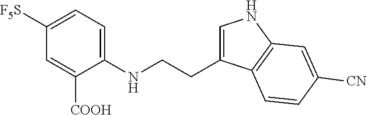

9.8% yield over five steps. Colorless solid. m.p. 210-212° C. ¹H NMR (400 MHz, DMSO-d₆) δ 13.31 (s, 1H), 11.50 (d, J=2.6 Hz, 1H), 8.39 (s, 1H), 8.15 (d, J=2.8 Hz, 1H), 7.85 (s, 1H), 7.84-7.75 (m, 2H), 7.57 (s, 1H), 7.31 (d, J=8.3 Hz, 1H), 6.92 (d, J=9.5 Hz, 1H), 3.58 (q, J=6.2 Hz, 2H), 3.07 (t, J=7.0 Hz, 2H).

### Example 39. Synthesis of

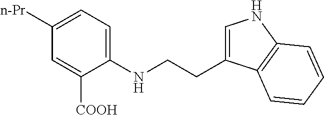

39% yield. Light yellow solid. ¹H NMR (400 MHz, DMSO-d₆) δ 10.85 (s, 1H), 7.59-7.55 (m, 2H), 7.34 (d, J=8.1 Hz, 1H), 7.24-7.19 (m, 2H), 7.07 (t, J=6.9 Hz, 1H), 6.97 (t, J=6.9 Hz, 1H), 6.72 (d, J=8.7 Hz, 1H), 3.44 (t, J=7.1 Hz, 2H), 3.01 (t, J=7.0 Hz, 2H), 2.41 (t, J=7.5 Hz, 2H), 1.51 (h, J=7.2 Hz, 2H), 0.86 (t, J=7.3 Hz, 3H).

### Example 40. Synthesis of

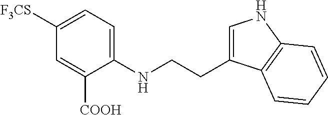

57% yield. Light yellow solid. ¹H NMR (400 MHz, acetone-d₆) δ 10.43 (s, 1H), 7.88 (s, 1H), 7.58 (s, 1H), 7.20-7.10 (m, 2H), 6.90 (d, J=8.1 Hz, 1H), 6.78 (s, 1H), 6.63 (t, J=7.6 Hz, 1H), 6.53 (t, J=6.9 Hz, 1H), 6.48 (d, J=9.0 Hz, 1H), 3.09 (t, J=7.1 Hz, 2H), 2.60 (t, J=7.0 Hz, 2H).

### Example 41. Synthesis of

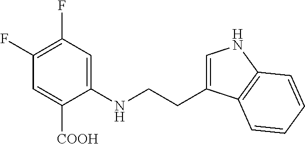 [SMILES: O=C(O)c1cc(F)c(F)cc1NCCc1c[nH]c2ccccc12]

37% yield. Light yellow solid. ¹H NMR (400 MHz, DMSO-d₆) δ 10.86 (s, 1H), 7.71-7.65 (m, 1H), 7.57 (d, J=7.9 Hz, 1H), 7.34 (d, J=8.1 Hz, 1H), 7.22 (d, J=2.4 Hz, 1H), 7.07 (t, J=6.9 Hz, 1H), 6.98 (t, J=7.5 Hz, 1H), 6.83-6.75 (m, 1H), 3.45 (t, J=7.0 Hz, 2H), 3.02 (t, J=7.0 Hz, 2H).

### Example 42. Synthesis of

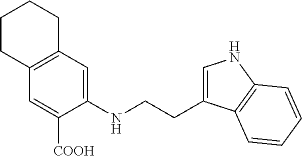 [SMILES: O=C(O)c1cc2c(cc1NCCc1c[nH]c3ccccc13)CCCC2]

34% yield. White solid. ¹H NMR (400 MHz, CDCl₃) δ 8.06 (s, 1H), 7.77 (s, 1H), 7.65 (d, J=7.9 Hz, 1H), 7.38 (d, J=8.4 Hz, 1H), 7.28 (s, 1H), 7.23-7.12 (m, 2H), 6.83 (s, 1H), 3.58 (t, J=6.9 Hz, 2H), 3.20 (t, J=6.9 Hz, 2H), 2.52 (t, J=7.6 Hz, 2H), 1.68-1.55 (m, 2H), 0.99-0.93 (m, 4H).

### Example 43. Synthesis of

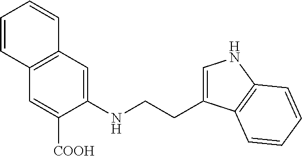 [SMILES: O=C(O)c1cc2ccccc2cc1NCCc1c[nH]c2ccccc12]

11% yield. Light yellow solid. ¹H NMR (400 MHz, DMSO-d₆) δ 8.47 (s, 1H), 7.77 (d, J=8.1 Hz, 1H), 7.62 (d, J=8.1 Hz, 2H), 7.40 (t, J=6.8 Hz, 1H), 7.35 (d, J=8.1 Hz, 1H), 7.26 (s, 1H), 7.13 (t, J=6.9 Hz, 1H), 7.08 (t, J=7.6 Hz, 1H), 6.99 (t, J=7.5 Hz, 1H), 6.95 (s, 1H), 3.52 (t, J=7.0 Hz, 2H), 3.11 (t, J=6.9 Hz, 2H).

### Example 44. Synthesis of

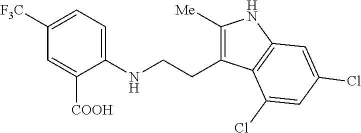 [SMILES: Cc1[nH]c2cc(Cl)cc(Cl)c2c1CCNc1ccc(C(F)(F)F)cc1C(=O)O]

43% yield. Light yellow solid. ¹H NMR (400 MHz, acetone-d₆) δ 10.44 (s, 1H), 8.36 (s, 1H), 8.18 (s, 1H), 7.61 (d, J=11.3 Hz, 1H), 7.32 (s, 1H), 7.04 (s, 1H), 6.99 (d, J=8.9 Hz, 1H), 3.65 (q, J=6.8 Hz, 2H), 3.29 (t, J=6.9 Hz, 2H), 2.41 (s, 3H).

### Example 45. Synthesis of

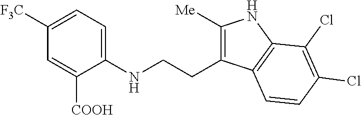 [SMILES: Cc1[nH]c2c(Cl)c(Cl)ccc2c1CCNc1ccc(C(F)(F)F)cc1C(=O)O]

48% yield. Light yellow solid. ¹H NMR (400 MHz, acetone-d₆) δ 10.36 (s, 1H), 8.31 (s, 1H), 8.17 (s, 1H), 7.61 (s, 1H), 7.52 (d, J=8.5 Hz, 1H), 7.14 (d, J=8.4 Hz, 1H), 6.98 (d, J=8.7 Hz, 1H), 3.61 (q, J=6.8 Hz, 2H), 3.12 (t, J=6.9 Hz, 2H), 2.46 (s, 3H).

### Example 46. Synthesis of

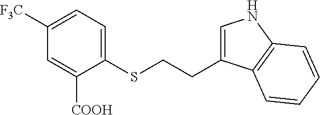 [SMILES: O=C(O)c1cc(C(F)(F)F)ccc1SCCc1c[nH]c2ccccc12]

A mixture of 2-bromo-5-(trifluoromethyl)benzoic acid (250 mg, 0.92 mmol), 2-(1H-indol-3-yl)ethane-1-thiol (330 mg, 1.9 mmol), Cs₂CO₃ (300 mg, 0.93 mmol), Cu powder (12 mg, 0.19 mmol), Cu₂O (13 mg, 0.092 mmol) and ethylene glycol diethyl ether (5 mL) was heated to 130° C. overnight under nitrogen. The reaction mixture was cooled to room temperature and the solvent was removed under reduced pressure. This residue was poured into H₂O (10 mL). The resulting solution was acidified with concentrated HCl (1M) to a pH of 2-3.

The resulting precipitate was filtered and washed with H₂O. The solid was redissolved in EtOAc and was purified by silica flash column chromatography with a gradient of EtOAc in hexanes of 0-100% over 7 min, eluting around 60% EtOAc as the free carboxylic acid. The solvent was removed by rotary evaporation under reduced pressure and isolated as the final product 46. 47% yield. Light yellow solid. ¹H NMR (400 MHz, acetone-d₆) δ 10.11 (s, 1H), 8.31 (s, 1H), 7.58 (dt, J=17.7, 8.1 Hz, 3H), 7.40 (d, J=7.9 Hz, 1H), 7.29 (s, 1H), 7.11 (t, J=7.0 Hz, 1H), 7.03 (t, J=7.6 Hz, 1H), 3.26 (t, J=8.1, 2H), 3.15 (t, J=7.0, 2H).

### Example 47. Synthesis of

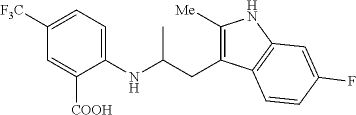 [SMILES: Cc1[nH]c2cc(F)ccc2c1CC(C)Nc1ccc(C(F)(F)F)cc1C(=O)O]

52% yield. Light yellow solid. ¹H NMR (400 MHz, acetone-d₆) δ 11.32 (s, 1H), 10.16 (s, 1H), 8.42 (d, J=7.5 Hz, 1H), 8.18 (s, 1H), 7.63-7.58 (m, 2H), 7.25 (s, 1H), 7.11 (d, J=12.4 Hz, 1H), 7.02 (d, J=9.0 Hz, 1H), 6.87-6.80 (m, 1H), 4.16-4.08 (m, 1H), 3.14-3.01 (m, 2H), 1.31 (d, J=6.4 Hz, 3H).

### Example 48. Synthesis of

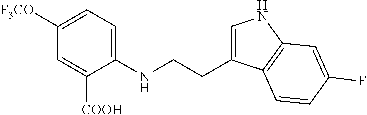 [SMILES: O=C(O)c1cc(OC(F)(F)F)ccc1NCCc1c[nH]c2cc(F)ccc12]

41% yield. White solid. ¹H NMR (400 MHz, DMSO-d₆) δ 10.95 (s, 1H), 7.64 (s, 1H), 7.61-7.54 (m, 1H), 7.39 (d, J=9.8 Hz, 1H), 7.23 (s, 1H), 7.12 (d, J=12.5 Hz, 1H), 6.91-6.79 (m, 2H), 3.50 (t, J=7.0 Hz, 2H), 3.02 (t, J=7.1 Hz, 2H).

### Example 49. Synthesis of

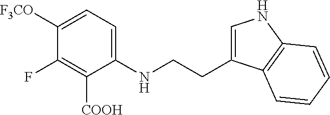 [SMILES: O=C(O)c1c(NCCc2c[nH]c3ccccc23)ccc(OC(F)(F)F)c1F]

43% yield. Light yellow solid. ¹H NMR (400 MHz, DMSO-d₆) δ 10.89 (s, 1H), 8.04 (s, 1H), 7.63-7.52 (m, 2H), 7.35 (d, J=8.1 Hz, 1H), 7.23 (s, 1H), 7.08 (t, J=6.9 Hz, 1H), 6.99 (t, J=6.9 Hz, 1H), 6.70 (d, J=9.2 Hz, 1H), 3.53 (t, J=7.6 Hz, 2H), 3.03 (t, J=7.0 Hz, 2H).

### Examples 50-61

The compounds of Examples 50-61 can be prepared as described by Faber, E. B., et al., J. Med. Chem. 2023, 66, 1928-1940. DOI: 10.1021/acs.jmedchem.2c01731

Anthranilic acids 50, 51, 52, 56, 57, 58, 59, 60, and 60 were prepared using procedures similar to those described herein above.

### General Ullmann Coupling to Yield Compounds 53, 54, and 55

The respective 2-bromobenzoic acid (0.9 mmol), tryptamine (1.5 equiv), potassium phosphate (2 equiv), copper (I) iodide (0.1 equiv), [1,1′-binaphthalene]-2,2′-diol (0.2 equiv) were added to anhydrous DMF (8 mL) and stirred at rt for 24 hours under nitrogen. The reaction was then filtered through Celite®, whereby 1M HCl (aq) was added dropwise until pH<4. The product was extracted three times into EtOAc. The EtOAc layer was then dried with MgSO₄, filtered, and concentrated under reduced pressure. The remaining residue was purified by silica flash column chromatography with a gradient of EtOAc in hexanes of 0-100% over 7 minutes, eluting around 60% EtOAc as the free carboxylic acid. The solvent was removed by rotary evaporation to furnish the final product.

### Example 50. Synthesis of 2-((2-(1H-Indol-3-yl)ethyl)amino)-3-nitrobenzoic acid

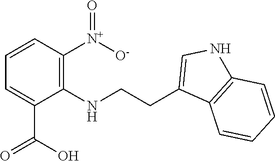 [SMILES: O=C(O)c1cccc([N+](=O)[O-])c1NCCc1c[nH]c2ccccc12]

47% yield. Yellow solid. m.p. 75-77° C. ¹H NMR (400 MHz, DMSO-d₆) δ 10.87 (s, 1H), 8.70 (s, 1H), 8.05 (d, J=7.6 Hz, 1H), 7.96 (d, J=8.1 Hz, 1H), 7.54 (d, J=7.9 Hz, 1H), 7.34 (d, J=8.0 Hz, 1H), 7.16 (s, 1H), 7.08 (t, J=7.5 Hz, 1H), 6.98 (t, J=7.4 Hz, 1H), 6.73 (t, J=7.9 Hz, 1H), 3.16-3.09 (m, 2H), 3.00 (t, J=6.8 Hz, 2H).

### Example 51. Synthesis of 2-((2-(1H-Indol-3-yl)ethyl)amino)-3-methyl-5-nitrobenzoic acid

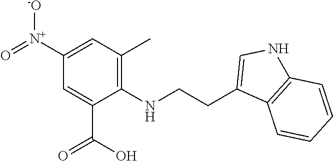

81% yield. Yellow solid. m.p. 210-211° C. ¹H NMR (400 MHz, DMSO-d₆) δ 13.40 (s, 1H), 10.86 (s, 1H), 8.47 (d, J=2.9 Hz, 1H), 8.01 (d, J=2.9 Hz, 1H), 7.56 (d, J=7.9 Hz, 1H), 7.34 (d, J=8.0 Hz, 1H), 7.18 (s, 1H), 7.07 (t, J=7.6 Hz, 1H), 6.98 (t, J=7.4 Hz, 1H), 3.82-3.77 (m, 2H), 3.00 (t, J=7.0 Hz, 2H), 2.43 (s, 3H).

### Example 52. Synthesis of 2-((2-(1H-Indol-3-yl)ethyl)amino)-4-bromobenzoic acid

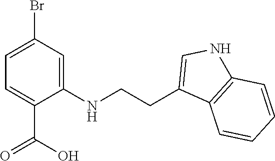 [SMILES: O=C(O)c1ccc(Br)cc1NCCc1c[nH]c2ccccc12]

66% yield. Colorless solid. m.p. 201-202° C. ¹H NMR (400 MHz, DMSO-d₆) δ 12.77 (s, 1H), 10.87 (s, 1H), 8.01 (s, 1H), 7.67 (d, J=8.4 Hz, 1H), 7.58 (d, J=7.9 Hz, 1H), 7.35 (d, J=8.1 Hz, 1H), 7.23 (s, 1H), 7.08 (t, J=7.8 Hz, 1H), 6.98 (t, J=7.4 Hz, 1H), 6.93 (s, 1H), 6.70 (dd, J=8.4, 1.9 Hz, 1H), 3.48 (t, J=7.0 Hz, 2H), 3.03 (t, J=6.9 Hz, 2H). ¹³C NMR (101 MHz, DMSO-d₆) δ 169.4, 151.5, 136.3, 133.4, 128.6, 127.0, 123.1, 121.0, 118.3, 116.8, 113.4, 111.4, 111.2, 109.0, 42.7, 24.4.

### Example 53. Synthesis of 2-((2-(1H-Indol-3-yl)ethyl)amino)-5-(methoxycarbonyl)benzoic acid

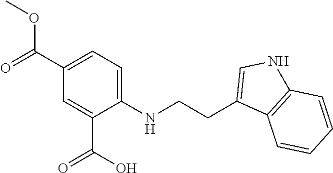 [SMILES: COC(=O)c1ccc(NCCc2c[nH]c3ccccc23)c(C(=O)O)c1]

56% yield. Colorless solid. m.p. 233-234° C. ¹H NMR (400 MHz, acetone-d₆) δ 10.09 (s, 1H), 8.63 (s, 1H), 8.55 (s, 1H), 7.98 (d, J=9.0 Hz, 1H), 7.66 (d, J=7.8 Hz, 1H), 7.40 (d, J=7.8 Hz, 1H), 7.28 (s, 1H), 7.12 (t, J=7.6 Hz, 1H), 7.04 (t, J=8.0 Hz, 1H), 6.91 (d, J=9.0 Hz, 1H), 3.83 (s, 3H), 3.70-3.63 (m, 2H), 3.18 (t, J=7.1 Hz, 2H). ¹³C NMR (101 MHz, acetone-d₆) δ 170.0, 166.8, 155.1, 137.8, 136.1, 135.4, 128.4, 123.8, 122.2, 119.5, 119.3, 116.5, 112.8, 112.2, 111.9, 109.8, 51.8, 44.1, 25.6.

### Example 54. Synthesis of 2-((2-(1H-Indol-3-yl)ethyl)amino)-5-acetylbenzoic acid

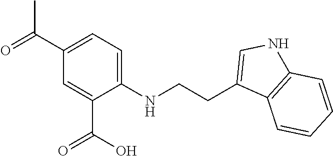 [SMILES: CC(=O)c1ccc(NCCc2c[nH]c3ccccc23)c(C(=O)O)c1]

54% yield. Light yellow solid. m.p. 228-229° C. ¹H NMR (400 MHz, acetone-d₆) δ 10.09 (s, 1H), 8.60 (s, 1H), 8.00 (d, J=9.0 Hz, 1H), 7.66 (d, J=8.9 Hz, 1H), 7.40 (d, J=7.5 Hz, 1H), 7.28 (s, 1H), 7.12 (t, J=7.6 Hz, 1H), 7.04 (t, J=7.4 Hz, 1H), 6.92 (d, J=9.0 Hz, 1H), 3.68 (q, J=6.7 Hz, 2H), 3.15-3.21 (m, 2H), 2.48 (s, 3H). ¹³C NMR (101 MHz, acetone-d₆) δ 195.1, 170.2, 155.0, 137.8, 135.3, 134.6, 128.4, 125.1, 123.8, 122.2, 119.5, 119.3, 112.8, 111.8, 109.6, 44.1, 25.9, 25.7.

### Example 55. Synthesis of 2-((2-(1H-Indol-3-yl)ethyl)amino)-5-(trifluoromethoxy)benzoic acid

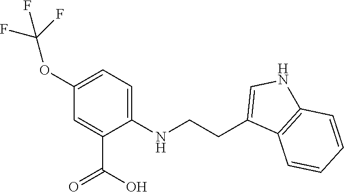 [SMILES: O=C(O)c1cc(OC(F)(F)F)ccc1NCCc1c[nH]c2ccccc12]

78% yield. Colorless solid. m.p. 177-179° C. ¹H NMR (400 MHz, acetone-d₆) δ 11.22 (s, 1H), 10.05 (s, 1H), 8.07 (s, 1H), 7.78 (s, 1H), 7.64 (s, 1H), 7.38 (t, J=9.0 Hz, 2H), 7.27 (s, 1H), 7.11 (t, J=7.6 Hz, 1H), 7.02 (t, J=7.4 Hz, 1H), 6.93 (d, J=9.3 Hz, 1H), 3.58-3.64 (m, 2H), 3.16 (t, J=7.1 Hz, 2H). ¹⁹F NMR (376 MHz, acetone-d₆) δ 118.1. ¹³C NMR (101 MHz, acetone-d₆) δ 169.3, 151.3, 137.8, 129.1, 128.4, 125.2, 123.7, 122.2, 110.1, 119.5, 119.3, 113.4, 113.0, 112.2, 110.2, 44.3, 25.7.

### Example 56. Synthesis of 2-((2-(1-Methyl-1H-indol-3-yl)ethyl)amino)-5-nitrobenzoic acid

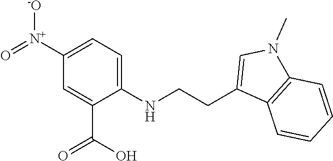

44% yield. Yellow solid. m.p. 220-223° C. ¹H NMR (400 MHz, DMSO-d₆) δ 13.45 (s, 1H), 8.87 (s, 1H), 8.63 (d, J=2.8 Hz, 1H), 8.18 (dd, J=9.4, 2.9 Hz, 1H), 7.61 (d, J=7.9 Hz, 1H), 7.40 (d, J=8.2 Hz, 1H), 7.24 (s, 1H), 7.15 (t, J=7.1 Hz, 1H), 7.02 (t, J=8.0 Hz, 1H), 6.96 (d, J=9.5 Hz, 1H), 3.75 (s, 3H), 3.64 (q, J=6.7 Hz, 2H), 3.06 (t, J=7.0 Hz, 2H). ¹³C NMR (101 MHz, DMSO-d₆) δ 168.6, 154.5, 136.7, 134.5, 129.4, 128.6, 127.6, 127.3, 121.2, 118.6, 118.4, 111.6, 110.2, 109.6, 109.3, 43.1, 32.3, 24.1.

### Example 57. Synthesis of 2-((2-(2-Methyl-1H-indol-3-yl)ethyl)amino)-5-nitrobenzoic acid

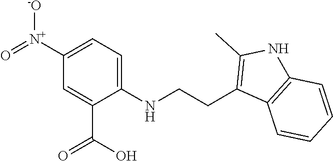

79% yield. Yellow solid. m.p. 119-120° C. ¹H NMR (400 MHz, DMSO-d₆) δ 13.44 (s, 1H), 10.76 (s, 1H), 8.78 (s, 1H), 8.61 (d, J=2.9 Hz, 1H), 8.14 (dd, J=9.4, 2.9 Hz, 1H), 7.48 (d, J=7.8 Hz, 1H), 7.23 (d, J=7.8 Hz, 1H), 6.99 (t, J=6.8 Hz, 1H), 6.93 (t, J=6.8 Hz, 1H), 6.88 (d, J=9.5 Hz, 1H), 3.61-3.52 (m, 2H), 3.01 (t, J=6.7 Hz, 2H), 2.34 (s, 3H). ¹³C NMR (101 MHz, DMSO-d₆) δ 168.6, 154.5, 135.3, 134.4, 132.8, 129.3, 128.6, 128.0, 120.1, 118.2, 117.3, 111.6, 110.4, 106.5, 42.9, 23.2, 11.2.

### Example 58. Synthesis of 2-((2-(5-Methyl-1H-indol-3-yl)ethyl)amino)-5-nitrobenzoic acid

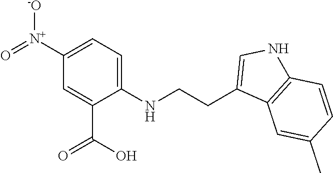

33% yield. Yellow solid. m.p. 247-251° C. ¹H NMR (400 MHz, DMSO-d₆) δ 13.42 (s, 1H), 10.74 (s, 1H), 8.87 (s, 1H), 8.63 (d, J=2.9 Hz, 1H), 8.17 (dd, J=9.5, 2.8 Hz, 1H), 7.35 (s, 1H), 7.23 (d, J=8.2 Hz, 1H), 7.19 (s, 1H), 6.96 (d, J=9.5 Hz, 1H), 6.90 (d, J=9.1 Hz, 1H), 3.63 (q, J=6.6 Hz, 2H), 3.03 (t, J=6.8 Hz, 2H), 2.37 (s, 3H).

### Example 59. Synthesis of 2-((2-(7-Methyl-1H-indol-3-yl)ethyl)amino)-5-nitrobenzoic acid

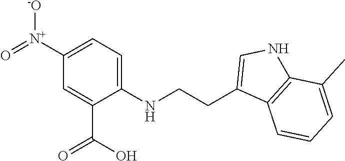

47% yield. Yellow solid. m.p. 253-257° C. ¹H NMR (400 MHz, DMSO-d₆) δ 13.42 (s, 1H), 10.86 (s, 1H), 8.86 (s, 1H), 8.63 (d, J=2.8 Hz, 1H), 8.18 (dd, J=9.5, 2.9 Hz, 1H), 7.42 (d, J=7.2 Hz, 1H), 7.24 (s, 1H), 6.96 (d, J=9.6 Hz, 1H), 6.92-6.86 (m, 2H), 3.67-3.61 (m, 2H), 3.06 (t, J=6.9 Hz, 2H), 2.44 (s, 3H).

### Example 60. Synthesis of 2-((2-(7-Chloro-1H-indol-3-yl)ethyl)amino)-5-nitrobenzoic acid

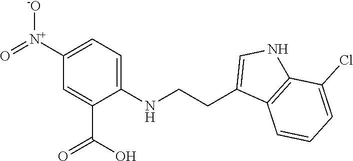

37% yield. Yellow solid. m.p. 275-279° C. ¹H NMR (400 MHz, DMSO-d₆) δ 13.43 (s, 1H), 11.28 (s, 1H), 8.86 (s, 1H), 8.63 (d, J=2.8 Hz, 1H), 8.18 (dd, J=9.5, 2.8 Hz, 1H), 7.59 (d, J=7.8 Hz, 1H), 7.34 (s, 1H), 7.17 (d, J=7.5 Hz, 1H), 7.04-6.94 (m, 2H), 3.66 (q, J=6.7 Hz, 2H), 3.08 (t, J=6.9 Hz, 2H). ¹³C NMR (101 MHz, DMSO-d₆) δ 168.6, 154.5, 134.5, 133.0, 129.4, 129.0, 128.6, 124.7, 120.6, 119.4, 117.5, 115.8, 112.4, 111.7, 42.9, 24.2.

### Example 61. Synthesis of 2-((2-(6-Methyl-1H-indol-3-yl)ethyl)amino)-5-nitrobenzoic acid

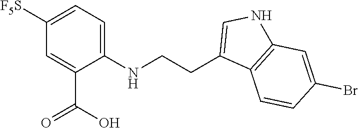

48% yield. Yellow solid. m.p. 222-224° C. ¹H NMR (400 MHz, DMSO-d₆) δ 13.42 (s, 1H), 10.72 (s, 1H), 8.85 (s, 1H), 8.63 (d, J=2.9 Hz, 1H), 8.18 (dd, J=9.4, 2.9 Hz, 1H), 7.47 (d, J=8.1 Hz, 1H), 7.17-7.11 (m, 2H), 6.95 (d, J=9.5 Hz, 1H), 6.82 (dd, J=8.1, 1.5 Hz, 1H), 3.63 (q, J=6.7 Hz, 2H), 3.03 (t, J=7.0 Hz, 2H), 2.38 (s, 3H).

### Examples 62-85

Unless otherwise described, the following General Coupling Procedure, which was adapted from Liu S, et al., Synthesis (Stuttg), 2007 Nov. 16, (22), 3519-3527 was used to prepare Examples 62-85

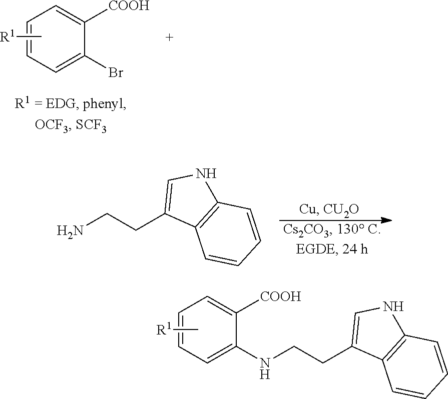

A mixture of 2-bromo-5-(trifluoromethyl)benzoic acid (250 mg, 0.92 mmol), 2-(1H-indol-3-yl)ethane-1-thiol (330 mg, 1.9 mmol), Cs₂CO₃ (300 mg, 0.93 mmol), Cu powder (12 mg, 0.19 mmol), Cu₂O (13 mg, 0.092 mmol) and ethylene glycol diethyl ether (5 mL) was heated to 130° C. overnight under nitrogen. The reaction mixture was cooled to room temperature and the solvent was removed under reduced pressure. This residue was poured into H₂O (10 mL). The resulting solution was acidified with concentrated HCl (1M) to a pH of 2-3. The resulting precipitate was filtered and washed with H₂O. The solid was redissolved in EtOAc and was purified by silica flash column chromatography with a gradient of EtOAc in hexanes of 0-100% over 7 min, eluting around 60% EtOAc as the free carboxylic acid. The solvent was removed by rotary evaporation under reduced pressure to obtain the final product.

### Example 62. Synthesis of 2-((2-(6-Cyano-1H-indol-3-yl)ethyl)amino)-4,5-diethoxybenzoic acid

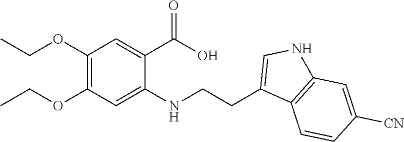 [SMILES: CCOc1cc(NCCc2c[nH]c3cc(C#N)ccc23)c(C(=O)O)cc1OCC]

Yield: 48%. Yellow solid. m.p. 178.6-179.7° C. ¹H NMR (400 MHz, acetone-d₆) δ 10.62 (s, 1H), 7.87-7.81 (m, 2H), 7.56 (s, 1H), 7.43 (s, 1H), 7.33 (d, J=8.2 Hz, 1H), 6.29 (s, 1H), 4.04 (q, J=7.0 Hz, 2H), 3.95 (q, J=7.0 Hz, 2H), 3.58 (t, J=7.0 Hz, 2H), 3.17 (t, J=7.0 Hz, 2H), 1.39 (t, J=7.0 Hz, 3H), 1.32 (t, J=7.0 Hz, 3H). Purity: 97.26%.

### Example 63. Synthesis of 2-((2-(6-Bromo-1H-indol-3-yl)ethyl)amino)-5-(trifluoromethoxy)benzoic acid

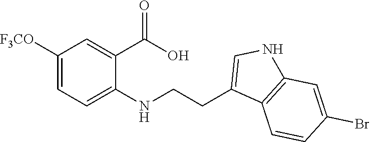 [SMILES: O=C(O)c1cc(OC(F)(F)F)ccc1NCCc1c[nH]c2cc(Br)ccc12]

57% yield. White solid. m.p. 210.2-210.7° C. ¹H NMR (400 MHz, DMSO-d₆) δ 11.03 (s, 1H), 7.64 (s, 1H), 7.55 (d, J=8.4 Hz, 1H), 7.53 (s, 1H), 7.38 (d, J=12.2 Hz, 1H), 7.27 (s, 1H), 7.10 (d, J=8.4 Hz, 1H), 6.87 (d, J=9.3 Hz, 1H), 3.49 (t, J=7.0 Hz, 2H), 3.02 (t, J=7.0 Hz, 2H). ¹⁹F NMR (376 MHz, DMSO-d6) δ −57.59. Purity: 97.09%.

### Synthesis of Amine Building Blocks for Examples 64 and 65

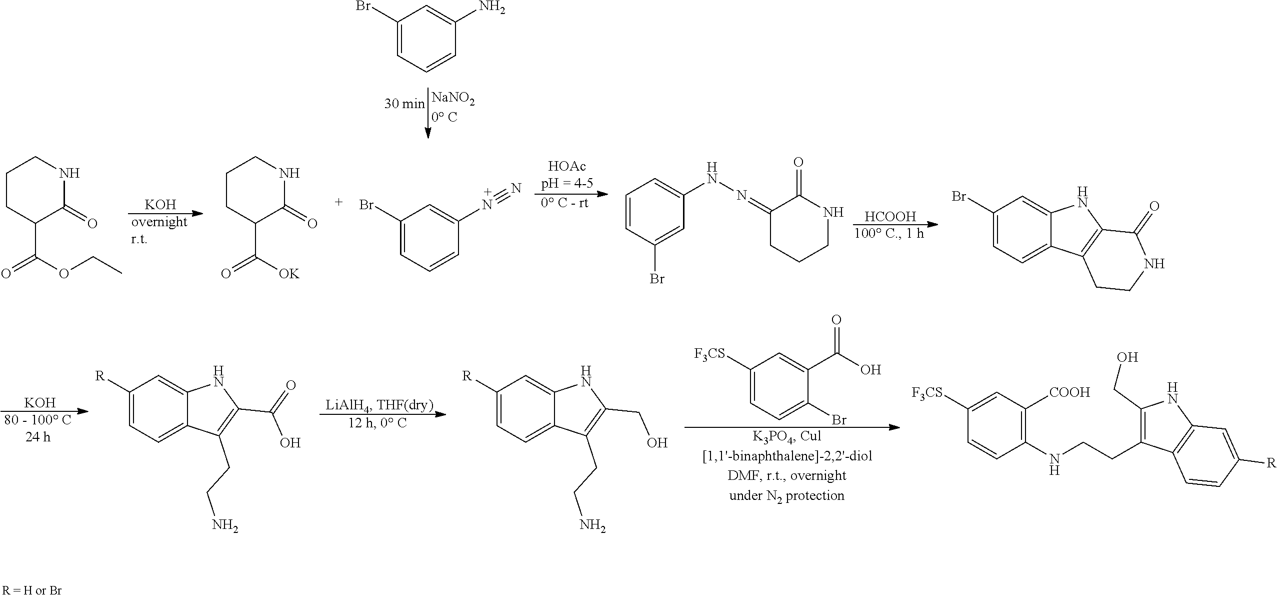

3-Carb ethoxy-2-piperidone (4.7 g, 27.4 mmol) was stirred with potassium hydroxide (1.64 g) in water (56 mL) and kept at 30° C. on an oil bath overnight. 3-Bromoaniline (4.99 g, 29 mmol) was treated with water (50 mL) and concentrated HCl (10 mL) and cooled to 0° C. Sodium nitrite (2.46 g, 35 mmol) in water (9 mL) was added dropwise to the above solution at 0° C. and stirred for an additional 20 minutes. Urea (7 mmol) was added to decompose the excess nitrous acid, and the diazotized solution was neutralized with 10% aqueous sodium carbonate. The resulting solution was filtered into the solution of previously hydrolyzed 3-carbethoxy-2-piperidone (2-piperidone-3-carboxylic acid) at 0° C. After a few minutes, acetic acid was added to adjust the pH of the mixture to 3-4. The reaction mixture was stirred at 0° C. for 5-6 hours, and the yellow precipitate which resulted was filtered, washed with water, and dried to get 3-(2-(3-bromophenyl)hydrazineylidene)piperidin-2-one (2.5 g, 32% yield). ¹H NMR (400 MHz, DMSO) δ 9.75 (s, 1H), 7.91 (t, J=3.2 Hz, 1H), 7.46 (d, J=1.8 Hz, 1H), 7.25-7.16 (m, 2H), 6.99 (d, J=6.6 Hz, 1H), 3.20 (t, J=7.2 Hz, 2H), 2.57 (t, J=6.6 Hz, 2H), 1.86 (p, J=6.4 Hz, 2H).

A mixture of 3-(2-(3-bromophenyl)hydrazineylidene)piperidin-2-one (3.04 g) and formic acid (40.0 mL) was stirred at 100° C. for 1 hour. The reaction mixture was cooled to room temperature and then concentrated under reduced pressure, and the obtained residue was purified by silica gel column chromatography (DCM:ethanol=0:0 to 10:1) to obtain 7-bromo-2,3,4,9-tetrahydro-1H-pyrido[3,4-b]indol-1-one (1.92 g, 67%) as a pale grey solid. ¹H NMR (400 MHz, DMSO-d₆) δ 11.77 (s, 1H), 7.65 (s, 1H), 7.58 (d, J=8.5 Hz, 1H), 7.55 (s, 1H), 7.20 (d, J=8.5 Hz, 1H), 3.52 (t, J=2.4 Hz, 3H), 2.92 (t, J=7.0 Hz, 2H).

To a mixture of 7-bromo-2,3,4,9-tetrahydro-1H-pyrido[3,4-b]indol-1-one (780 mg), ethanol (5.00 mL), and water (5.00 mL) was added potassium hydroxide (1.58 g), followed by stirring at 80° C. overnight. Potassium hydroxide (1.58 g) was added to the reaction mixture, followed by stirring at 100° C. for 6 hours. After cooling to room temperature, the solvent was evaporated under reduced pressure. The obtained residue was dissolved in water (10.0 mL). To the mixture was added 1M HCl to adjust the pH to 9-10, and the precipitated solid was collected by filtration and dried under reduced pressure to obtain 3-(2-aminoethyl)-6-bromo-1H-indole-2-carboxylic acid (607 mg, 73%) as a reddish brown solid. ¹H NMR (400 MHz, DMSO-d₆) δ 11.14 (s, 1H), 8.71 (s, 2H), 7.52 (d, J=8.5 Hz, 1H), 7.50 (s, 1H), 7.10 (d, J=8.5 Hz, 1H), 3.19 (t, J=5.9 Hz, 2H), 3.01 (t, J=5.8 Hz, 2H).

3-(2-Aminoethyl)-1H-indol-2-yl)methanol (0.1 mmol) was dissolved in anhydrous tetrahydrofuran (5 mL) under nitrogen. Then LiAlH₄ (4 equiv.) was added dropwise at 0° C. The solution was stirred at 0° C. for 4-6 hours then the reaction mixture was allowed to warm to room temperature and then stirred overnight. The reaction was quenched at 0° C. with water then NaOH solution. After the precipitation was formed, the mixture was filtered under vacuum though Celite. The filtered solvent was removed by rotary evaporation without further purification to produce the crude intermediate.

### Example 64. Synthesis of 2-((2-(2-(Hydroxymethyl)-1H-indol-3-yl)ethyl)amino)-5-((trifluoromethyl)thio)benzoic acid

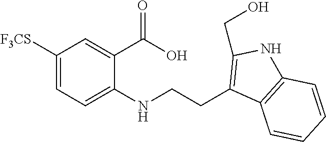

Yield: 2.4% over two steps. White solid. m.p. 209.2-210.2° C. ¹H NMR (400 MHz, DMSO-d₆) δ 10.89 (s, 1H), 8.02 (s, 1H), 7.57 (d, J=10.8 Hz, 1H), 7.54 (d, J=8.1 Hz, 1H), 7.30 (d, J=8.2 Hz, 1H), 7.03 (t, J=7.0 Hz, 1H), 6.95 (t, J=7.4 Hz, 1H), 6.88 (d, J=10.1 Hz, 1H), 4.62 (s, 2H), 3.45 (s, 2H), 3.03 (t, J=6.7 Hz, 2H). Purity: 96.96%.

### Example 65. Synthesis of

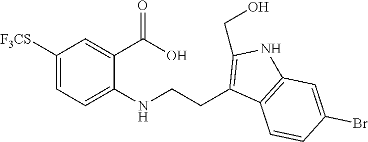

2-((2-(6-Bromo-2-(hydroxymethyl)-1H-indol-3-yl)ethyl)amino)-5-((trifluoromethyl)thio)benzoic acid (WN.3.121). Yield: 0.4% over two steps. White solid. m.p. 212.1-213.4° C. ¹H NMR (400 MHz, acetone-d₆) δ 10.69 (s, 1H), 8.97 (s, 1H), 8.25 (s, 1H), 7.55-7.43 (m, 3H), 7.07 (d, J=8.4 Hz, 1H), 6.77 (d, J=8.7 Hz, 1H), 5.36 (t, J=4.9 Hz, 1H), 4.80 (s, 2H), 3.53 (bs, 2H), 3.13 (t, J=6.5 Hz, 2H). Purity: 96.23%.

### Example 66. Synthesis of 2-((2-(6-Bromo-1H-indol-3-yl)ethyl)amino)-5-((trifluoromethyl)thio)benzoic acid

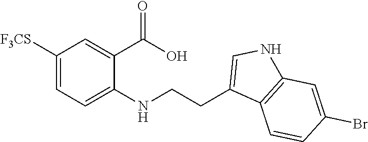

Yield: 69%. White solid. m.p. 208.5-209.6° C. ¹H NMR (400 MHz, acetone-d₆) δ 11.04 (s, 1H), 10.01 (s, 1H), 8.18 (s, 1H), 7.97 (s, 1H), 7.44-7.35 (m, 3H), 7.09 (s, 1H), 6.93 (d, J=8.5 Hz, 1H), 6.75 (d, J=9.0 Hz, 1H), 3.47-3.37 (m, 2H), 2.94 (t, J=7.1 Hz, 2H). Purity: 95.28%.

### Example 67. Synthesis of 2-((2-(6-Cyano-1H-indol-3-yl)ethyl)amino)-5-((trifluoromethyl)thio)benzoic acid

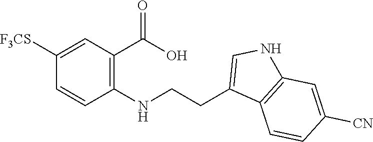

49% yield. Light yellow solid. m.p. 221.1-222.6° C. ¹H NMR (400 MHz, acetone-d₆) δ 10.70 (s, 1H), 8.42 (s, 1H), 8.20 (s, 1H), 7.88-7.82 (m, 2H), 7.66-7.59 (m, 2H), 7.33 (d, J=9.8 Hz, 1H), 6.98 (d, J=8.9 Hz, 1H), 3.72-3.64 (m, 2H), 3.22 (t, J=7.0 Hz, 2H). Purity: 96.84%.

### Example 68. Synthesis of 6-((2-(1H-Indol-3-yl)ethyl)amino)-2-methyl-3-nitrobenzoic

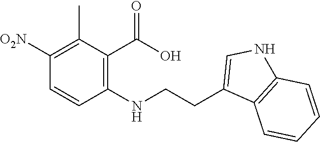 [SMILES: Cc1c([N+](=O)[O-])ccc(NCCc2c[nH]c3ccccc23)c1C(=O)O]

43% yield. White solid. m.p. 163.8-164.7° C. ¹H NMR (400 MHz, acetone-d₆) δ 11.91 (s, 1H), 10.08 (s, 1H), 7.99 (d, J=9.3 Hz, 1H), 7.65 (d, J=8.1 Hz, 1H), 7.41 (d, J=7.9 Hz, 1H), 7.27 (s, 1H), 7.12 (t, J=8.3 Hz, 1H), 7.07-7.00 (m, 1H), 6.83 (d, J=9.3 Hz, 1H), 6.76 (s, 1H), 3.65 (t, J=7.2 Hz, 2H), 3.16 (t, J=7.1 Hz, 2H), 2.58 (s, 3H). Purity: 96.33%.

### Example 69. Synthesis of 2-((2-(1H-Indol-3-yl)ethyl)amino)-5-bromo-4-methoxybenzoic acid

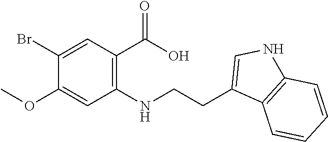 [SMILES: COc1cc(NCCc2c[nH]c3ccccc23)c(C(=O)O)cc1Br]

56% yield. Light yellow solid. m.p. 157.5-158.2° C. ¹H NMR (400 MHz, acetone-d₆) δ 10.07 (s, 1H), 8.21 (s, 1H), 8.00 (s, 1H), 7.66 (d, J=7.8 Hz, 1H), 7.40 (d, J=8.1 Hz, 1H), 7.26 (d, J=2.4 Hz, 1H), 7.12 (t, J=6.9 Hz, 1H), 7.07-6.99 (m, 1H), 6.34 (s, 1H), 3.85 (s, 3H), 3.61 (q, J=6.9 Hz, 2H), 3.16 (t, J=7.0 Hz, 2H). Purity: 95.59%.

### Example 70. Synthesis of 2-((2-(1H-Indol-3-yl)ethyl)amino)-4-chloro-5-methylbenzoic acid

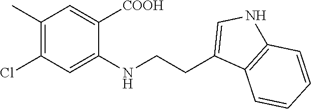 [SMILES: Cc1cc(C(=O)O)c(NCCc2c[nH]c3ccccc23)cc1Cl]

47% yield. Light yellow solid. m.p. 196.0-197.8° C. ¹H NMR (400 MHz, DMSO-d₆) δ 10.86 (s, 1H), 7.70 (s, 1H), 7.57 (d, J=7.9 Hz, 1H), 7.34 (d, J=8.0 Hz, 1H), 7.22 (s, 1H), 7.07 (t, J=6.9 Hz, 1H), 6.98 (t, J=6.9 Hz, 1H), 6.80 (s, 1H), 3.44 (t, J=6.9 Hz, 2H), 3.01 (t, J=7.0 Hz, 2H), 2.18 (s, 3H). Purity: 96.29%.

### Example 71. Synthesis of 2-((2-(1H-Indol-3-yl)ethyl)amino)-4,5-dimethoxybenzoic acid

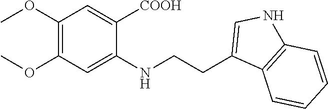 [SMILES: COc1cc(NCCc2c[nH]c3ccccc23)c(C(=O)O)cc1OC]

49% yield. White solid. m.p. 165.0-167.8° C. ¹H NMR (400 MHz, DMSO-d₆) δ 10.84 (s, 1H), 7.59 (d, J=7.9 Hz, 1H), 7.34 (d, J=8.1 Hz, 1H), 7.23 (s, 1H), 7.20 (s, 1H), 7.07 (t, J=7.6 Hz, 1H), 6.98 (t, J=7.5 Hz, 1H), 6.23 (s, 1H), 3.76 (s, 3H), 3.63 (s, 3H), 3.47 (t, J=7.1 Hz, 2H), 3.02 (t, J=6.9 Hz, 2H). Purity: 95.92%.

### Example 72. Synthesis of 6-((2-(1H-Indol-3-yl)ethyl)amino)benzo[d][1,3]dioxole-5-carboxylic acid

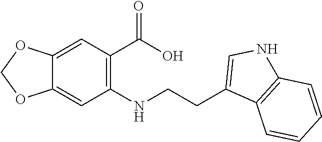 [SMILES: O=C(O)c1cc2c(cc1NCCc1c[nH]c3ccccc13)OCO2]

36% yield. Pale solid. m.p. 182.0-183.1° C. ¹H NMR (400 MHz, DMSO-d₆) δ 10.85 (s, 1H), 7.57 (d, J=7.9 Hz, 1H), 7.34 (d, J=8.1 Hz, 1H), 7.21 (s, 1H), 7.18 (s, 1H), 7.07 (t, J=7.5 Hz, 1H), 6.98 (t, J=7.5 Hz, 1H), 6.45 (s, 1H), 5.95 (s, 2H), 3.44 (t, J=7.0 Hz, 2H), 3.00 (t, J=7.0 Hz, 2H). Purity: 95.96%.

### Example 73. Synthesis of 7-((2-(1H-Indol-3-yl)ethyl)amino)-2,3-dihydrobenzo[b][1,4]dioxine-6-carboxylic acid

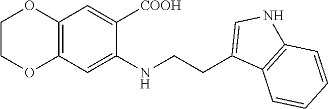 [SMILES: O=C(O)c1cc2c(cc1NCCc1c[nH]c3ccccc13)OCCO2]

54% yield. White solid. m.p. 182.9-183.8° C. ¹H NMR (400 MHz, DMSO-d₆) δ 10.84 (s, 1H), 7.56 (d, J=7.8 Hz, 1H), 7.34 (d, J=8.1 Hz, 1H), 7.23 (s, 1H), 7.20 (s, 1H), 7.06 (t, J=7.4 Hz, 1H), 6.97 (t, J=7.6 Hz, 1H), 6.19 (s, 1H), 4.26 (bs, 2H), 4.15 (bs, 2H), 3.38 (bs, 2H), 3.00 (d, J=6.9 Hz, 2H). Purity: 97.10%.

### Example 74. Synthesis of 2-((2-(1H-Indol-3-yl)ethyl)amino)-5-cyclopropylbenzoic acid

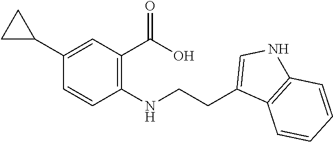 [SMILES: O=C(O)c1cc(C2CC2)ccc1NCCc1c[nH]c2ccccc12]

39% yield. White solid. m.p. 200.5-201.7° C. ¹H NMR (400 MHz, acetone-d₆) δ 10.76 (s, 1H), 10.04 (s, 1H), 7.69 (s, 1H), 7.65 (d, J=7.9 Hz, 1H), 7.40 (d, J=8.1 Hz, 1H), 7.26 (s, 1H), 7.17 (d, J=8.9 Hz, 1H), 7.11 (t, J=8.2 Hz, 1H), 7.03 (d, J=13.9 Hz, 1H), 6.78 (d, J=8.7 Hz, 1H), 3.55 (t, J=7.2 Hz, 2H), 3.13 (t, J=7.2 Hz, 2H), 1.89-1.80 (m, 1H), 0.90-0.81 (m, 2H), 0.61-0.52 (m, 2H). Purity: 95.53%.

### Example 75. Synthesis of 2-((2-(1H-Indol-3-yl)ethyl)amino)-5-methoxybenzoic acid

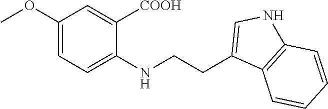 [SMILES: COc1ccc(NCCc2c[nH]c3ccccc23)c(C(=O)O)c1]

89% yield. Yellow solid. m.p. 170-173° C. ¹H NMR (400 MHz, acetone-d₆) δ 10.05 (s, 1H), 7.64 (d, J=7.8 Hz, 1H), 7.45 (s, 1H), 7.39 (d, J=7.5 Hz, 1H), 7.25 (s, 1H), 7.13-7.07 (m, 2H), 7.03 (d, J=7.9 Hz, 1H), 6.83 (d, J=9.0 Hz, 1H), 3.74 (s, 3H), 3.54 (t, J=7.2 Hz, 2H), 3.12 (t, J=7.2 Hz, 2H). ¹³C NMR (101 MHz, acetone-d₆) δ 170.1, 150.1, 147.6, 137.8, 128.5, 123.9, 123.6, 122.1, 119.4, 119.3, 115.7, 113.6, 113.4, 112.2, 110.4, 56.0, 44.6, 26.0.

### Example 76. Synthesis of 2-((2-(1H-Indol-3-yl)ethyl)amino)-5-methylbenzoic acid

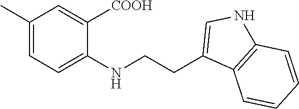 [SMILES: Cc1ccc(NCCc2c[nH]c3ccccc23)c(C(=O)O)c1]

42% yield. Light yellow solid. m.p. 186.8-187.3° C. ¹H NMR (400 MHz, acetone-d₆) δ 10.05 (s, 1H), 7.71 (s, 1H), 7.64 (d, J=7.9 Hz, 1H), 7.39 (d, J=8.1 Hz, 1H), 7.25 (s, 1H), 7.22 (d, J=8.5 Hz, 1H), 7.10 (t, J=6.9 Hz, 1H), 7.02 (t, J=6.9 Hz, 1H), 6.77 (d, J=8.5 Hz, 1H), 3.54 (t, J=7.2 Hz, 2H), 3.12 (t, J=7.1 Hz, 2H), 2.21 (s, 3H). Purity: 95.23%

### Example 77. Synthesis of 2-((2-(1H-Indol-3-yl)ethyl)amino)-5-ethylbenzoic acid

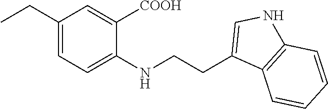 [SMILES: CCc1ccc(NCCc2c[nH]c3ccccc23)c(C(=O)O)c1]

41% yield. Light yellow solid. m.p. 203.1-204.2° C. ¹H NMR (400 MHz, acetone-d₆) δ 10.04 (s, 1H), 7.74 (s, 1H), 7.64 (d, J=7.9 Hz, 1H), 7.38 (d, J=8.1 Hz, 1H), 7.29-7.23 (m, 2H), 7.09 (t, J=6.9 Hz, 1H), 7.01 (t, J=6.9 Hz, 1H), 6.78 (d, J=8.5 Hz, 1H), 3.54 (t, J=7.2 Hz, 2H), 3.12 (t, J=6.8 Hz, 2H), 2.52 (q, J=7.6 Hz, 2H), 1.17 (t, J=7.6 Hz, 3H). Purity: 95.23%.

### Example 78. Synthesis of 2-((2-(1H-Indol-3-yl)ethyl)amino)-5-isopropylbenzoic acid

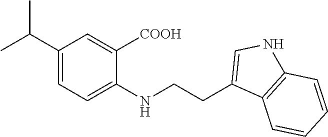 [SMILES: CC(C)c1ccc(NCCc2c[nH]c3ccccc23)c(C(=O)O)c1]

37% yield. Light yellow solid. m.p. 212.0-213.1° C. ¹H NMR (400 MHz, DMSO-d₆) δ 10.85 (s, 1H), 7.62 (s, 1H), 7.58 (d, J=7.8 Hz, 1H), 7.35 (d, J=8.1 Hz, 1H), 7.29 (d, J=8.7 Hz, 1H), 7.22 (s, 1H), 7.07 (t, J=6.9 Hz, 1H), 6.98 (t, J=6.9 Hz, 1H), 6.75 (d, J=8.7 Hz, 1H), 3.45 (t, J=7.0 Hz, 2H), 3.02 (t, J=7.1 Hz, 2H), 2.77 (p, J=6.9 Hz, 1H), 1.15 (d, J=7.0 Hz, 6H). Purity: 95.25%.

### Example 79. Synthesis of 4-((2-(1H-Indol-3-yl)ethyl)amino)-[1,1′-biphenyl]-3-carboxylic acid

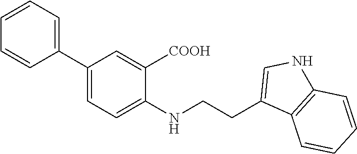 [SMILES: O=C(O)c1cc(-c2ccccc2)ccc1NCCc1c[nH]c2ccccc12]

22% yield. Light yellow solid. m.p. 156.2-157.7° C. ¹H NMR (400 MHz, acetone-d₆) δ 10.08 (s, 1H), 8.24 (s, 1H), 7.75 (d, J=8.9 Hz, 1H), 7.68 (d, J=8.2 Hz, 1H), 7.61 (d, J=8.2 Hz, 2H), 7.46-7.39 (m, 3H), 7.31-7.26 (m, 2H), 7.12 (t, J=7.6 Hz, 1H), 7.04 (t, J=8.0 Hz, 1H), 6.98 (d, J=8.7 Hz, 1H), 3.65 (t, J=7.1 Hz, 2H), 3.19 (t, J=7.2 Hz, 2H). Purity: 97%.

### Example 80. Synthesis of 2-((2-(1H-Indol-3-yl)ethyl)amino)-5-(tert-butyl)benzoic acid

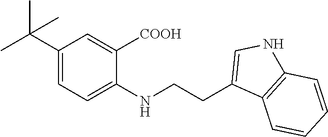 [SMILES: CC(C)(C)c1ccc(NCCc2c[nH]c3ccccc23)c(C(=O)O)c1]

57% yield. Light yellow solid. m.p. 228.1-229.4° C. ¹H NMR (400 MHz, acetone-d₆) δ 10.04 (s, 1H), 7.94 (s, 1H), 7.64 (d, J=7.0 Hz, 1H), 7.49 (d, J=8.7 Hz, 1H), 7.38 (d, J=8.1 Hz, 1H), 7.25 (s, 1H), 7.09 (t, J=7.6 Hz, 1H), 7.01 (t, J=7.4 Hz, 1H), 6.81 (d, J=8.9 Hz, 1H), 3.55 (t, J=7.2 Hz, 2H), 3.12 (t, J=7.2 Hz, 2H), 1.28 (s, 9H). Purity: 95.29%.

### Example 81. Synthesis of 2-((2-(1H-Indol-3-yl)ethyl)amino)-5-propylbenzoic acid

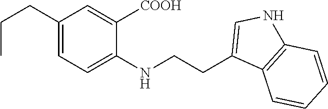 [SMILES: CCCc1ccc(NCCc2c[nH]c3ccccc23)c(C(=O)O)c1]

39% yield. Light yellow solid. m.p. 204.1-205.6° C. ¹H NMR (400 MHz, DMSO-d₆) δ 10.85 (s, 1H), 7.59-7.55 (m, 2H), 7.34 (d, J=8.1 Hz, 1H), 7.24-7.19 (m, 2H), 7.07 (t, J=6.9 Hz, 1H), 6.97 (t, J=6.9 Hz, 1H), 6.72 (d, J=8.7 Hz, 1H), 3.44 (t, J=7.1 Hz, 2H), 3.01 (t, J=7.0 Hz, 2H), 2.41 (t, J=7.5 Hz, 2H), 1.51 (h, J=7.2 Hz, 2H), 0.86 (t, J=7.3 Hz, 3H). Purity: 96.11%.

### Example 82. Synthesis of 2-((2-(1H-Indol-3-yl)ethyl)amino)-5-isopropoxybenzoic acid

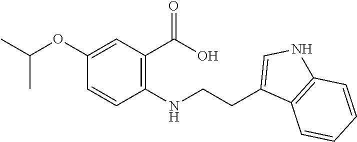 [SMILES: CC(C)Oc1ccc(NCCc2c[nH]c3ccccc23)c(C(=O)O)c1]

49% yield. Light yellow solid. m.p. 182.0-183.5° C. ¹H NMR (400 MHz, acetone-d₆) δ 10.06 (s, 1H), 7.65 (d, J=7.2 Hz, 1H), 7.47 (s, 1H), 7.40 (d, J=8.1 Hz, 1H), 7.26 (s, 1H), 7.14-7.06 (m, 2H), 7.03 (t, J=6.9 Hz, 1H), 6.81 (d, J=9.2 Hz, 1H), 4.41 (hept, J=6.0 Hz, 1H), 3.54 (t, J=7.2 Hz, 2H), 3.13 (t, J=7.2 Hz, 2H), 1.26 (d, J=6.1 Hz, 6H). Purity: 97.47%.

### Example 83. Synthesis of 2-((2-(1H-Indol-3-yl)ethyl)amino)-5-isopropoxybenzoic acid

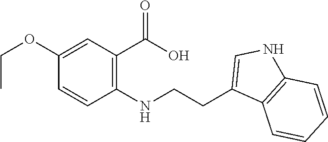 [SMILES: CCOc1ccc(NCCc2c[nH]c3ccccc23)c(C(=O)O)c1]

58% yield. Yellow solid. m.p. 176.3-177.4° C. ¹H NMR (400 MHz, DMSO-d₆) δ 10.85 (s, 1H), 7.57 (d, J=7.8 Hz, 1H), 7.34 (d, J=8.2 Hz, 1H), 7.29 (s, 1H), 7.21 (s, 1H), 7.10-7.04 (m, 2H), 6.97 (t, J=7.6 Hz, 1H), 6.75 (d, J=9.2 Hz, 1H), 3.91 (q, J=6.9 Hz, 2H), 3.43 (t, J=7.0 Hz, 2H), 3.01 (t, J=7.0 Hz, 2H), 1.28 (t, J=6.9 Hz, 3H). Purity: 97.06%.

### Example 84. Synthesis of 2-((2-(1H-Indol-3-yl)ethyl)amino)-5-(tert-butoxy)benzoic acid

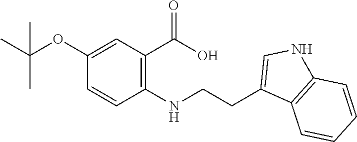 [SMILES: CC(C)(C)Oc1ccc(NCCc2c[nH]c3ccccc23)c(C(=O)O)c1]

58% yield. White solid. m.p. 200.2-201.4° C. ¹H NMR (400 MHz, acetone-d₆) δ 10.06 (s, 1H), 7.65 (d, J=7.8 Hz, 1H), 7.54 (d, J=2.9 Hz, 1H), 7.40 (d, J=8.1 Hz, 1H), 7.27 (s, 1H), 7.15-7.06 (m, 2H), 7.03 (t, J=8.0 Hz, 1H), 6.77 (d, J=9.0 Hz, 1H), 3.55 (t, J=7.2 Hz, 2H), 3.14 (t, J=7.2 Hz, 2H), 1.28 (s, 9H). Purity: 96.40%.

### Example 85. Synthesis of 2-((2-(1H-Indol-3-yl)ethyl)amino)-5-(ethylthio)benzoic acid

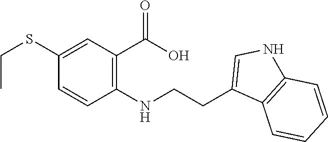 [SMILES: CCSc1ccc(NCCc2c[nH]c3ccccc23)c(C(=O)O)c1]

49% yield. White solid. m.p. 183.6-184.4° C. ¹H NMR (400 MHz, acetone-d₆) δ 11.03 (s, 1H), 10.08 (s, 1H), 8.09 (s, 1H), 8.02 (s, 1H), 7.66 (d, J=7.9 Hz, 1H), 7.49 (d, J=8.7 Hz, 1H), 7.40 (d, J=8.1 Hz, 1H), 7.27 (s, 1H), 7.11 (t, J=7.6 Hz, 1H), 7.03 (t, J=7.5 Hz, 1H), 6.86 (d, J=8.9 Hz, 1H), 3.59 (t, J=7.1 Hz, 2H), 3.15 (t, J=7.1 Hz, 2H), 2.77 (q, J=7.2 Hz, 2H), 1.19 (t, J=7.3 Hz, 3H). Purity: 96.71%.

Examples 86-93 were prepared using the General Synthetic Method For Preparing Compounds Wherein R¹ is COOR^{a} and X is NH described above.

### Example 86. Synthesis of 2-((2-(1H-Indol-3-yl)ethyl)amino)-4-bromobenzoic acid

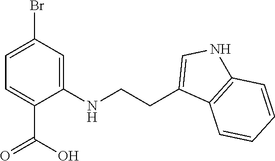 [SMILES: O=C(O)c1ccc(Br)cc1NCCc1c[nH]c2ccccc12]

66% yield. White solid. 1H NMR (400 MHz, DMSO-d₆) δ 12.77 (s, 1H), 10.87 (s, 1H), 8.01 (s, 1H), 7.67 (d, J=8.4 Hz, 1H), 7.58 (d, J=7.9 Hz, 1H), 7.35 (d, J=8.1 Hz, 1H), 7.23 (s, 1H), 7.08 (t, J=7.8 Hz, 1H), 6.98 (t, J=7.4 Hz, 1H), 6.93 (s, 1H), 6.70 (dd, J=8.4, 1.9 Hz, 1H), 3.48 (t, J=7.0 Hz, 2H), 3.03 (t, J=6.9 Hz, 2H). 13C NMR (101 MHz, DMSO) δ 169.4, 151.5, 136.3, 133.4, 128.6, 127.0, 123.1, 121.0, 118.3, 116.8, 113.4, 111.4, 111.2, 109.0, 42.7, 24.4.

### Example 87. Synthesis of 2-((2-(1H-Indol-3-yl)ethyl)amino)-4-amino-5-nitrobenzoic acid

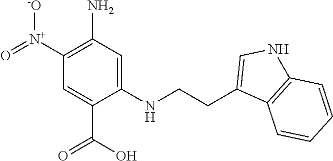

69% yield. Yellow solid. ¹H NMR (400 MHz, DMSO-d₆O) δ 12.81 (s, 1H), 10.89 (s, 1H), 8.61 (s, 1H), 8.39 (s, 1H), 7.61 (d, J=7.8 Hz, 1H), 7.54 (s, 2H), 7.35 (d, J=8.1 Hz, 1H), 7.24 (s, 1H), 7.08 (t, J=7.5 Hz, 1H), 6.99 (t, J=7.4 Hz, 1H), 6.05 (s, 1H), 3.45 (q, J=6.6 Hz, 2H), 3.06 (t, J=7.1 Hz, 2H). ¹³C NMR (101 MHz, DMSO) δ 168.4, 153.5, 149.9, 136.3, 132.6, 127.0, 123.1, 122.2, 121.0, 118.34, 118.31, 111.4, 111.1, 103.0, 93.2, 43.0, 24.0.

### Example 88. Synthesis of 2-((2-(1H-Indol-3-yl)ethyl)amino)-6-nitrobenzoic acid

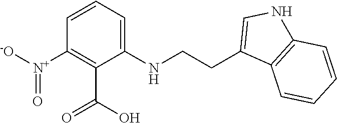

43% yield. Yellow solid. ¹H NMR (400 MHz, DMSO-d₆) δ 10.86 (s, 1H), 7.59 (d, J=7.9 Hz, 1H), 7.45 (t, J=8.2 Hz, 1H), 7.35 (d, J=8.1 Hz, 1H), 7.22 (s, 1H), 7.12-7.04 (m, 2H), 6.98 (t, J=7.5 Hz, 1H), 6.93 (d, J=7.7 Hz, 1H), 3.50 (t, J=6.9 Hz, 2H), 3.01 (t, J=7.1 Hz, 2H). ¹³C NMR (101 MHz, DMSO) δ 166.6, 151.7, 148.9, 136.3, 132.7, 127.0, 123.0, 121.0, 118.3, 115.3, 111.4, 111.1, 109.7, 43.2, 24.4.

### Example 89. Synthesis of 2-((2-(1H-Indol-3-yl)ethyl)amino)-6-(trifluoromethyl)benzoic acid

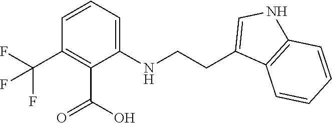 [SMILES: O=C(O)c1c(NCCc2c[nH]c3ccccc23)cccc1C(F)(F)F]

6% yield. White solid. ¹H NMR (400 MHz, DMSO-d₆) δ 10.85 (s, 1H), 7.59 (d, J=8.1 Hz, 1H), 7.43 (t, J=8.4 Hz, 1H), 7.36 (d, J=8.3 Hz, 1H), 7.21 (s, 1H), 7.12-7.03 (m, 2H), 7.00 (t, J=7.4 Hz, 1H), 6.94 (d, J=8.0 Hz, 1H), 3.49-3.40 (m, 2H), 3.00 (t, J=7.3 Hz, 2H). ¹⁹F NMR (376 MHz, DMSO) δ −57.6.

### Example 90. Synthesis of 2-((2-(1H-Indol-3-yl)ethyl)amino)-6-cyanobenzoic acid

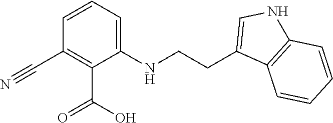 [SMILES: N#Cc1cccc(NCCc2c[nH]c3ccccc23)c1C(=O)O]

15% yield. Yellow solid. ¹H NMR (400 MHz, DMSO-d₆) δ 10.87 (s, 1H), 7.68 (t, J=7.8 Hz, 1H), 7.60 (d, J=7.8 Hz, 1H), 7.34 (d, J=8.1 Hz, 1H), 7.25 (s, 1H), 7.22 (d, J=8.6 Hz, 1H), 7.13 (d, J=7.1 Hz, 1H), 7.08 (t, J=7.5 Hz, 1H), 6.98 (t, J=7.4 Hz, 1H), 6.76 (t, J=5.9 Hz, 1H), 3.63 (q, J=6.8 Hz, 2H), 3.03 (t, J=7.2 Hz, 2H).

### Example 91. Synthesis of 2-((2-(1H-Indol-3-yl)ethyl)amino)-5-formylbenzoic acid

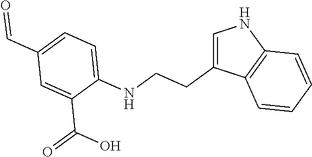 [SMILES: O=Cc1ccc(NCCc2c[nH]c3ccccc23)c(C(=O)O)c1]

18% yield. White solid. ¹H NMR (400 MHz, DMSO-d₆) δ 13.06 (s, 1H), 10.89 (s, 1H), 9.69 (s, 1H), 8.73 (s, 1H), 8.32 (s, 1H), 7.83 (d, J=8.9 Hz, 1H), 7.60 (d, J=7.9 Hz, 1H), 7.35 (d, J=8.1 Hz, 1H), 7.25 (s, 1H), 7.08 (t, J=7.5 Hz, 1H), 7.04-6.92 (m, 2H), 3.63-3.58 (m, 2H), 3.06 (t, J=7.0 Hz, 2H). ¹³C NMR (101 MHz, DMSO) δ 189.8, 169.3, 154.5, 136.3, 136.2, 134.0, 127.0, 123.5, 123.2, 123.0, 121.0, 118.3, 111.7, 111.4, 111.0, 109.6, 42.8, 24.4.

### Example 92. Synthesis of 2-(3-(2-((2-Carboxy-4-nitrophenyl)amino)ethyl)-1H-indol-2-yl)pyridin-1-ium chloride

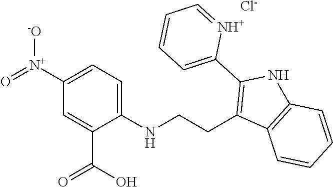

76% yield. Yellow solid. ¹H NMR (400 MHz, DMSO-d₆) δ 13.33 (s, 1H), 11.51 (s, 1H), 8.97 (s, 1H), 8.73 (d, J=4.7 Hz, 1H), 8.60 (d, J=2.8 Hz, 1H), 8.23 (dd, J=9.5, 2.8 Hz, 1H), 7.96-7.85 (m, 2H), 7.74 (d, J=7.9 Hz, 1H), 7.42 (d, J=8.1 Hz, 1H), 7.33 (t, J=6.8 Hz, 1H), 7.23 (d, J=9.5 Hz, 1H), 7.17 (t, J=7.5 Hz, 1H), 7.04 (t, J=7.4 Hz, 1H), 3.72-3.65 (m, 2H), 3.50 (t, J=7.7 Hz, 2H).

### Example 93. Synthesis of 3-(2-((2-Carboxy-4-nitrophenyl)amino)ethyl)-1H-indole-2-carboxylic acid

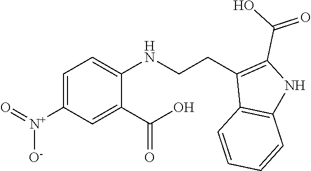 [SMILES: O=C(O)c1cc([N+](=O)[O-])ccc1NCCc1c(C(=O)O)[nH]c2ccccc12]

15% yield. Yellow solid. ¹H NMR (400 MHz, DMSO-d₆) δ 11.53 (s, 1H), 9.33 (s, 1H), 8.64 (d, J=2.9 Hz, 1H), 8.10 (dd, J=9.5, 2.8 Hz, 1H), 7.74 (d, J=8.1 Hz, 1H), 7.41 (d, J=8.2 Hz, 1H), 7.24 (t, J=7.7 Hz, 1H), 7.09-7.00 (m, 2H), 3.62-3.58 (m, 2H), 3.41 (t, J=7.5 Hz, 2H). ¹³C NMR (101 MHz, DMSO) δ 168.5, 163.3, 154.8, 136.0, 134.3, 128.7, 128.5, 127.3, 124.7, 124.6, 120.2, 119.4, 118.6, 112.4, 111.1, 43.3, 23.8.

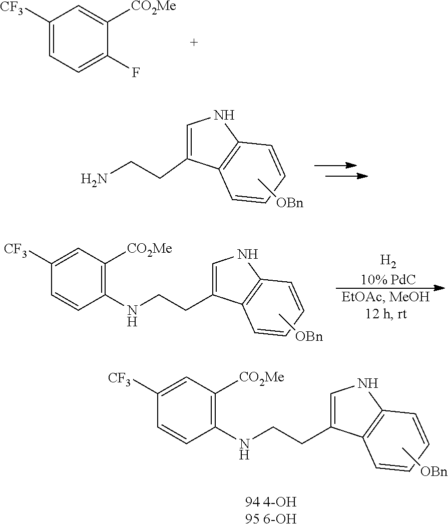

### Examples 94-95

Due to the stability of hydroxy substitutions at various positions around the indole ring, neutral pH conditions were used for the successful synthesis of these compounds. First, commercial benzyl protected tryptamine derivatives (0.40 mmol scale) were used to make the respective anthranilic acids using the General Synthetic Method For Preparing Compounds Wherein R¹ is COOR^{a} and X is NH described above to yield the intermediate acids. Next, the acids were dissolved in a 1:1 ratio of EtOAc:MeOH with 10% mol Pd/C. The solution was flushed with 1 atm H₂ and allowed to stir for 12 hours. The solution was loaded onto Celite and purified with RediSep Rf Gold Silica Gel Disposable Flash columns from Teledyne Isco (4 g, 18 mL/min from rate) with a gradient of MeOH in DCM of 0-20% in 5 min, eluting around 10% MeOH. The final products were isolated after rotary evaporation.

### Example 94. Synthesis of 2-((2-(4-Hydroxy-1H-indol-3-yl)ethyl)amino)-5-(trifluoromethyl)benzoic acid

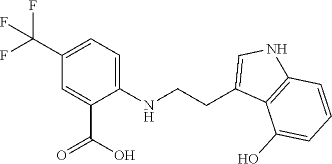 [SMILES: O=C(O)c1cc(C(F)(F)F)ccc1NCCc1c[nH]c2cccc(O)c12]

42% yield over 3 steps. White solid. ¹H NMR (400 MHz, DMSO-d₆) δ 13.09 (s, 1H), 10.68 (s, 1H), 9.42 (s, 1H), 8.36 (s, 1H), 8.01 (s, 1H), 7.61 (dd, J=9.0, 2.4 Hz, 1H), 7.06 (d, J=9.0 Hz, 1H), 7.00 (s, 1H), 6.87-6.75 (m, 2H), 6.34 (d, J=7.2 Hz, 1H), 3.55 (t, J=7.3 Hz, 2H), 3.11 (t, J=7.2 Hz, 2H). ¹⁹F NMR (376 MHz, DMSO) δ −59.6. ¹³C NMR (101 MHz, DMSO-d₆) δ 169.0, 152.9, 151.6, 138.7, 130.6, 128.8, 121.9, 121.6, 116.4, 113.8, 113.5, 112.0, 111.1, 109.0, 103.0, 102.8, 44.1, 26.1.

### Example 95. Synthesis of 2-((2-(6-Hydroxy-1H-indol-3-yl)ethyl)amino)-5-(trifluoromethyl)benzoic acid

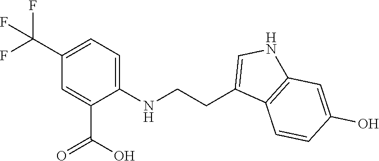 [SMILES: O=C(O)c1cc(C(F)(F)F)ccc1NCCc1c[nH]c2cc(O)ccc12]

19% yield over 3 steps. White solid. ¹H NMR (400 MHz, DMSO-d₆) δ 10.46 (s, 1H), 8.86 (s, 1H), 8.38 (s, 1H), 8.01 (s, 1H), 7.62 (dd, J=9.0, 2.4 Hz, 1H), 7.34 (d, J=8.5 Hz, 1H), 7.00 (s, 1H), 6.93 (d, J=9.0 Hz, 1H), 6.70 (s, 1H), 6.51 (dd, J=8.5, 2.1 Hz, 1H), 3.51 (t, J=7.1 Hz, 2H), 2.97 (t, J=7.0 Hz, 2H). ¹⁹F NMR (376 MHz, DMSO-d₆) δ −59.2. ¹³C NMR (101 MHz, DMSO-d₆) δ 168.9, 153.0, 152.7, 137.4, 130.6, 128.8, 120.9, 120.6, 118.6, 111.8, 111.0, 108.9, 96.5, 42.7, 24.5.

### Example 96. Biological Assays

### Protein Expression and Purification of CDK2

The expression and purification of CDK2 was carried out as described by: Schönbrunn, E, et al., in J Med Chem 2013, 56 (10), 3768-82).

p-CDK2 Expression and Purification and Cyclin a Expression and Purification

p-CDK2 expression and purification and cyclin A expression and purification was carried out as described by: A. Majumdar, D. J. Burban, J. M. Muretta, A. R. Thompson, T. A. Engel, D. M. Rasmussen, et al. Nature Chemical Biology 2021, 17, (4), 456-464.

p-Cl-ANS Displacement Assay

The p-Cl-ANS fluorescent binding assay was conducted as described previously (Faber, E. B., et al., ACS Chem Biol 2020, 15 (7), 1759-64). Compounds in DMSO (1% final) were added to 384-well black clear bottom plates (Greiner 781091) in 8-point dose response in duplicate using the Echo 550 (Beckman). SU9516 and DMSO were used as controls representing 100% and 0% inhibition of p-Cl-ANS binding, respectively. 20 μL of assay buffer (150 mM NaCl, 50 mM HEPES, pH 7.5 containing 0.01% Triton and 2 mM DTT) was added to each well, the plate was shaken for 1 minute, centrifuged at 2000 rpm for 5 min intervals until air bubbles were eliminated and then read on a CLARIOstar plate reader (BMG; Ex 388 nm, Em 455 nm) to obtain background fluorescence measurements for correction of intrinsic compound fluorescence. Absorbance was then measured at both the excitation and emission wavelengths for correction of the inner filter effect. Next, 5 μL p-Cl-ANS (Wang, N., et al., ACS Omega 2019, 4 (19), 18472-7; 15 μM final) was added to each well in assay buffer. Where present, staurosporine (Enzo Life Sciences; final 5 μM) was added as a premixed solution with p-Cl-ANS. Lastly, 5 uL of 3 μM CDK2 (final 0.5 μM) in buffer was added (final volume of 30 μL) and the plate was shaken, centrifuged, and read as described above. The data were analyzed by correcting the raw fluorescence data using the pre-read fluorescence and absorbance data to minimize test compound fluorescence interference and inner filter effect as described previously (Faber, E. B., et al., ACS Chem Biol 2020, 15 (7), 1759-64). IC₅₀ values were calculated using the four-parameter logistic equation using Prism 6.0 (GraphPad) and are expressed as the mean±SEM values of ≥3 independent experiments. Data for representative compounds of Formula (I) is provided below.

### Isothermal Titration Calorimetry (ITC)

CDK2 was thawed at room temperature and buffer exchanged using Zeba™ spin desalting columns. The final ITC buffer was 1×PBS, 10 mM MgCl₂, and 5% glycerol (pH=7.4), with 5% DMSO added after buffer exchange into the protein solution and from a 1:20 concentrated DMSO stock for the ligand solution. CDK2 concentration was determined using a NanoDrop™ spectrophotometer and calculated using absorption at 280 nm (ε=36,900 M⁻¹ cm⁻¹). Into a 96-well plate for automatic injection for the MicroCal iTC200 (Malvern), a final volume of 400 μL protein solution for the sample cell and 200 μL inhibitor solution for the injecting syringe were placed into the appropriate wells. The final inhibitor concentration in the syringe was 500-1,000 μM depending on compound solubility. For Compounds 3-7, to get more accurate data on the ITC with appropriate c values (Wiseman, T., et al., Anal. Biochem. 1989, 179 (1), 131-7), the CDK2 concentration was lowered about 10-fold to 5 μM and the syringe concentration lowered to 75 μM. The ITC experiments were conducted at 25° C. and 750 rpm stirring, with a discarded initial 0.4 μL injection and 20 subsequent 4 μL injections with 180 s between injections. A one-site binding model was used to fit the ITC data after adjusting the baseline to account for slight buffer mismatch between the cell and syringe samples. All ITC runs were done in duplicates (n=2) for each compound. Data for representative compounds of Formula (I) is provided in Table 1 below.

ADP Glo Experiments Kinase activity of phosphorylated CDK2 with cyclin A and varying amounts of inhibitor were measured using the ADP Glo Kinase Assay (Promega) in a luminescence plate reader (Enspire Multimode Plate Reader). Triplicate reactions were carried out in Kinase Reaction Buffer (50 mM HEPES, pH 7.5, 150 mM NaCl, 2 mM EGTA, 0.01% BSA, 10 mM MgCl₂, 1 mM DTT). Assays were performed with 2.5 nM phosphorylated CDK2, 200 μM ATP (Promega), 600 ng/well Histone H1 substrate (AnaSpec), and varying concentrations of cyclin A (5, 50, or 500 nM). Phosphorylated CDK2 was pre-incubated with inhibitor at 20° C. for 10 min. Reactions were initiated by simultaneously adding ATP and cyclin A and incubated at 20° C. for min and then quenched with ADP Glo reagent following the manufacturer's instruction. Luminescence was measured using a 1 s integration time and normalized to the no-inhibitor 5% DMSO controls. Kinase activity was determined by globally fitting relative luminescence data from all 3 cyclin A concentrations to a quadratic binding model using non-linear regression in GraphPad Prism 9. For fitting, CDK2 concentration was constrained to 2.5 nM with data sets from each CDK2:cyclin A ratio globally constrained to share the lower baseline parameter. IC₅₀ values reported represent the 95% confidence intervals obtained from the fits (n=3) using the symmetrical likelihood method in GraphPad Prism 9.

### Surface Plasmon Resonance (SPR) Methodology and Data

The SPR method used was largely adapted from a published method for CDK2 SPR studies (Wood, D. J., et al., Cell Chem Biol 2018, 26, 1-10. DOI: 10.1016/j.chembiol.2018.10.015). Briefly, a Biacore S200 (Cytiva) at 20° C. and CM5 chips were used for binding analysis. Multi-cycle runs were used for small molecule binding analysis, whereas a single-cycle run was used to measure cyclin binding. The buffer used to prepare the protein samples was 20 mM HEPES, 150 mM NaCl, 10 mM MgCl₂, 0.01% Tween 20, pH=7.4). For runs with small molecules, an additional 1% DMSO was added for solubility. GST capture kit conditions (Cytiva catalog number BR100223) were used to capture anti-GST antibody on both the sample and reference cells (7 min immobilization, 10 μL/min flow rate). Both surfaces had high affinity sites capped with an additional 3 min of GST flowed over (5 μg/mL concentration, 5 μL/min flow rate) followed by regeneration (10 mM glycine, pH=2.2). On the subtractive reference surface, GST was immobilized (20 μg/mL, 5 μL/min, 5 min). On the sample surface, GST-tagged CDK2 was immobilized in a similar fashion (50 μg/mL, 5 μL/min, 5 min).

For small molecule binding analysis, two startup cycles of running buffer were carried out on both surfaces after protein immobilization. Depending on observed binding kinetics, a 60-180 s association time and a 360 s dissociation time at 30 μL/min for each concentration of a particular compound over both surfaces was injected. An eight-point dose response was conducted with increasing concentrations for each compound with concentrations that were >10*K_{d} at the highest and <0.1*K_{d} at the lowest. This differed slightly for testing compound 4.1, which demonstrated nonspecific binding to the chip surface at concentrations >25 μM. Zero concentrations were performed at the beginning and end of the dose response as a baseline and to ensure internal reproducibility. An additional maximum concentration of compound was injected after the second zero concentration as an additional internal control. Lastly, a five-point DMSO solvent correction was performed for each compound before analysis with 0.6%, 0.8%, 1.0%, 1.2%, and 1.4% DMSO in buffer. To determine the K_{d} and kinetic binding parameters, the Biacore S200 Evaluation Software (Cytiva) using affinity fit, and when possible kinetic fit, was used.

### Results

Data for representative compounds of the invention in the p-Cl-ANS assay, the ITC assay, and the SPR assay is provided in the following Table 1.

| | | p-CI-ANS | ITC | SPR |
|---|---|---|---|---|
| Example | Structure | IC₅₀ (μM) | K_{d} (μM) | K_{d} (μM) |
| 1 | 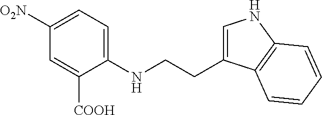 [SMILES: O=C(O)c1cc([N+](=O)[O-])ccc1NCCc1c[nH]c2ccccc12] | 4.6 | 4.9 | 1.8 (SSA) |
| 2 | 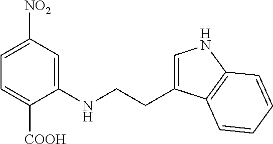 [SMILES: O=C(O)c1ccc([N+](=O)[O-])cc1NCCc1c[nH]c2ccccc12] | 6.1 | 13 | |
| 3 | 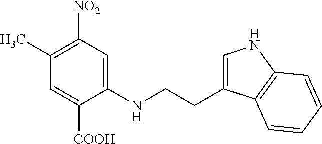 [SMILES: Cc1cc(C(=O)O)c(NCCc2c[nH]c3ccccc23)cc1[N+](=O)[O-]] | 21 | 9.7 | |
| 4 | 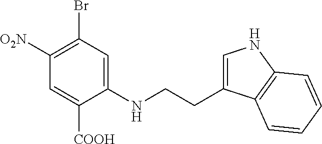 [SMILES: O=C(O)c1cc([N+](=O)[O-])c(Br)cc1NCCc1c[nH]c2ccccc12] | 2.8 | | |
| 5 | 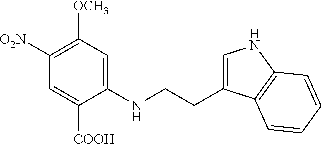 [SMILES: COc1cc(NCCc2c[nH]c3ccccc23)c(C(=O)O)cc1[N+](=O)[O-]] | 4.6 | | |
| 6 | 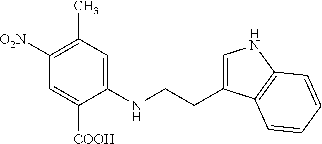 [SMILES: Cc1cc(NCCc2c[nH]c3ccccc23)c(C(=O)O)cc1[N+](=O)[O-]] | 4.2 | 5.7 | |
| 7 | 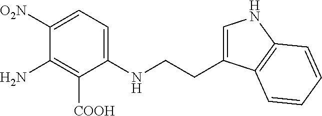 [SMILES: Nc1c([N+](=O)[O-])ccc(NCCc2c[nH]c3ccccc23)c1C(=O)O] | 4.1 | 4.4 | |
| 8 | 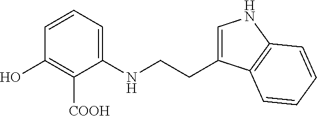 [SMILES: O=C(O)c1c(O)cccc1NCCc1c[nH]c2ccccc12] | 20 | 5.9 | |
| 9 | 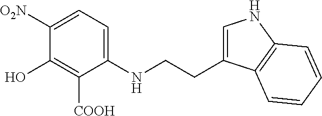 [SMILES: O=C(O)c1c(NCCc2c[nH]c3ccccc23)ccc([N+](=O)[O-])c1O] | 0.66 | 0.36 | |
| 10 | 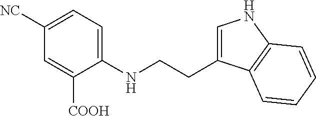 [SMILES: N#Cc1ccc(NCCc2c[nH]c3ccccc23)c(C(=O)O)c1] | 15 | 9.2 | |
| 11 | 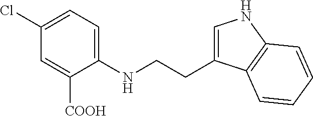 [SMILES: O=C(O)c1cc(Cl)ccc1NCCc1c[nH]c2ccccc12] | 8 | 8.2 | |
| 12 | 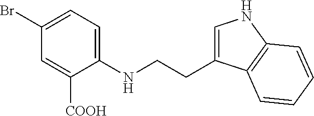 [SMILES: O=C(O)c1cc(Br)ccc1NCCc1c[nH]c2ccccc12] | 4 | 9.2 | |
| 13 | 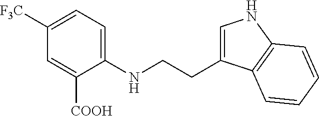 [SMILES: O=C(O)c1cc(C(F)(F)F)ccc1NCCc1c[nH]c2ccccc12] | 1.1 | 1.9 | |
| 14 | 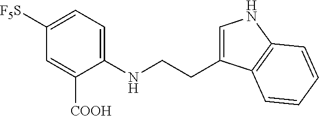 | 0.30 | 0.26 | |
| 15 | 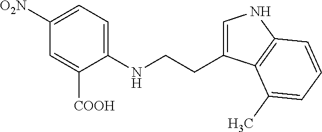 [SMILES: Cc1cccc2[nH]cc(CCNc3ccc([N+](=O)[O-])cc3C(=O)O)c12] | 5.0 | | |
| 16 | 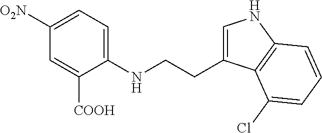 [SMILES: O=C(O)c1cc([N+](=O)[O-])ccc1NCCc1c[nH]c2cccc(Cl)c12] | 3.9 | | |
| 17 | 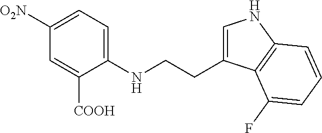 [SMILES: O=C(O)c1cc([N+](=O)[O-])ccc1NCCc1c[nH]c2cccc(F)c12] | 2.1 | | |
| 18 | 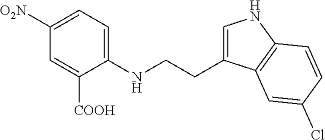 [SMILES: O=C(O)c1cc([N+](=O)[O-])ccc1NCCc1c[nH]c2ccc(Cl)cc12] | 5.8 | | |
| 19 | 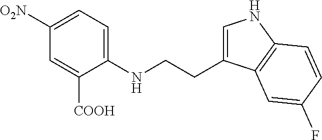 [SMILES: O=C(O)c1cc([N+](=O)[O-])ccc1NCCc1c[nH]c2ccc(F)cc12] | 2.5 | | |
| 20 | 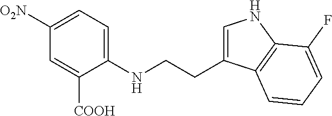 [SMILES: O=C(O)c1cc([N+](=O)[O-])ccc1NCCc1c[nH]c2c(F)cccc12] | 6.0 | | |
| 21 | 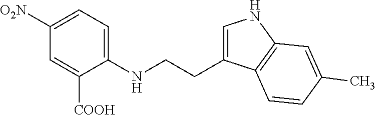 [SMILES: Cc1ccc2c(CCNc3ccc([N+](=O)[O-])cc3C(=O)O)c[nH]c2c1] | 0.92 | | |
| 22 | 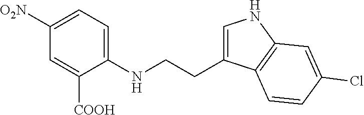 [SMILES: O=C(O)c1cc([N+](=O)[O-])ccc1NCCc1c[nH]c2cc(Cl)ccc12] | 0.44 | | |
| 23 | 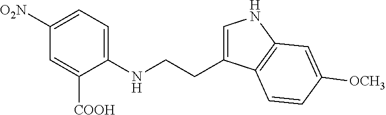 [SMILES: COc1ccc2c(CCNc3ccc([N+](=O)[O-])cc3C(=O)O)c[nH]c2c1] | 1.5 | | |
| 24 | 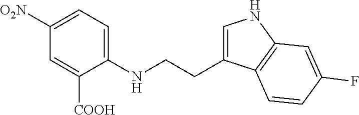 [SMILES: O=C(O)c1cc([N+](=O)[O-])ccc1NCCc1c[nH]c2cc(F)ccc12] | 0.37 | | |
| 25 | 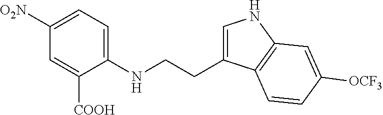 [SMILES: O=C(O)c1cc([N+](=O)[O-])ccc1NCCc1c[nH]c2cc(OC(F)(F)F)ccc12] | 0.94 | | |
| 26 | 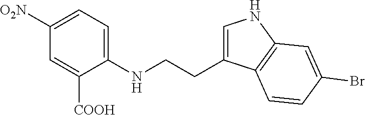 [SMILES: O=C(O)c1cc([N+](=O)[O-])ccc1NCCc1c[nH]c2cc(Br)ccc12] | 0.25 | | |
| 27 | 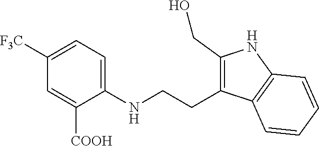 [SMILES: O=C(O)c1cc(C(F)(F)F)ccc1NCCc1c(CO)[nH]c2ccccc12] | 0.63 | | |
| 28 | 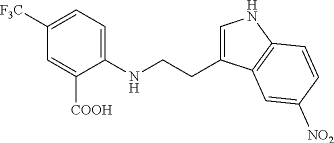 [SMILES: O=C(O)c1cc(C(F)(F)F)ccc1NCCc1c[nH]c2ccc([N+](=O)[O-])cc12] | 0.66 | | |
| 29 | 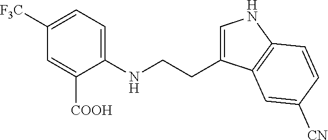 [SMILES: N#Cc1ccc2[nH]cc(CCNc3ccc(C(F)(F)F)cc3C(=O)O)c2c1] | 6.2 | | |
| 30 | 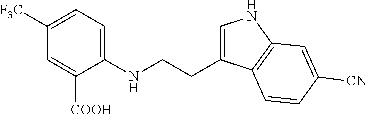 [SMILES: N#Cc1ccc2c(CCNc3ccc(C(F)(F)F)cc3C(=O)O)c[nH]c2c1] | 0.10 | 0.023 | 0.0014 (kinetic) 0.0065 (SSA) |
| 31 | 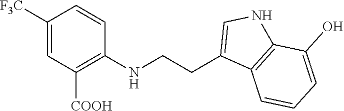 [SMILES: O=C(O)c1cc(C(F)(F)F)ccc1NCCc1c[nH]c2c(O)cccc12] | 3.4 | | |
| 32 | 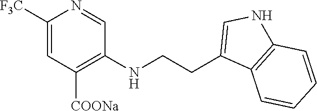 | 10 | | |
| 33 | 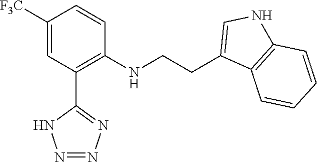 [SMILES: FC(F)(F)c1ccc(NCCc2c[nH]c3ccccc23)c(-c2nnn[nH]2)c1] | 6.6 | | |
| 34 | 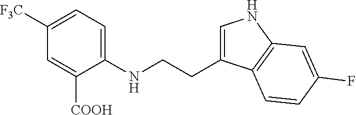 [SMILES: O=C(O)c1cc(C(F)(F)F)ccc1NCCc1c[nH]c2cc(F)ccc12] | 0.17 | 0.21 | |
| 35 | 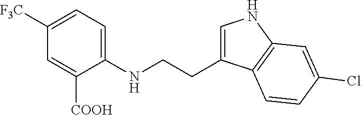 [SMILES: O=C(O)c1cc(C(F)(F)F)ccc1NCCc1c[nH]c2cc(Cl)ccc12] | 0.092 | 0.031 | 0.024 (SSA) |
| 36 | 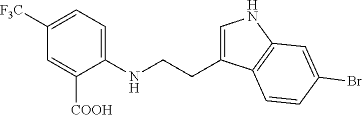 [SMILES: O=C(O)c1cc(C(F)(F)F)ccc1NCCc1c[nH]c2cc(Br)ccc12] | 0.16 | 0.018 | 0.020 (kinetic) 0.016 (SSA) |
| 37 | 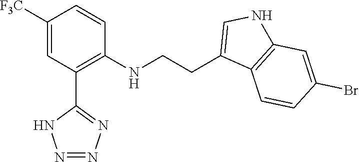 [SMILES: FC(F)(F)c1ccc(NCCc2c[nH]c3cc(Br)ccc23)c(-c2nnn[nH]2)c1] | 0.63 | | |
| 38 | 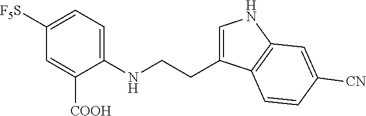 | 0.087 | 0.0074 | |
| 39 | 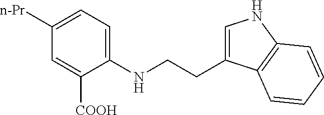 | 5.6 | 6.5 | |
| 40 | 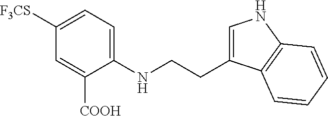 | 0.15 | | |
| 41 | 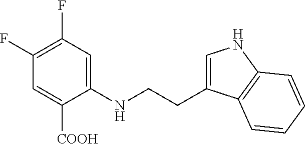 [SMILES: O=C(O)c1cc(F)c(F)cc1NCCc1c[nH]c2ccccc12] | 2.4 | | |
| 42 | 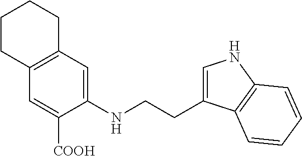 [SMILES: O=C(O)c1cc2c(cc1NCCc1c[nH]c3ccccc13)CCCC2] | 6.8 | | |
| 43 | 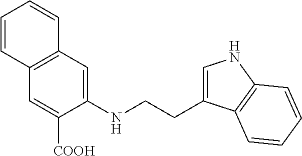 [SMILES: O=C(O)c1cc2ccccc2cc1NCCc1c[nH]c2ccccc12] | 2.7 | | |
| 44 | 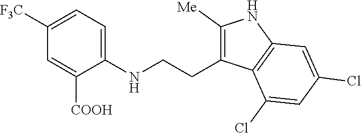 [SMILES: Cc1[nH]c2cc(Cl)cc(Cl)c2c1CCNc1ccc(C(F)(F)F)cc1C(=O)O] | 0.93 | | |
| 45 | 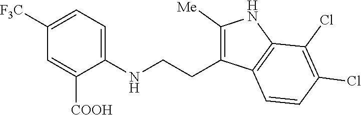 [SMILES: Cc1[nH]c2c(Cl)c(Cl)ccc2c1CCNc1ccc(C(F)(F)F)cc1C(=O)O] | 1.6 | | |
| 46 | 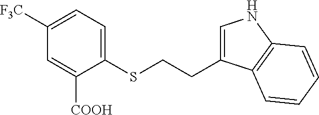 [SMILES: O=C(O)c1cc(C(F)(F)F)ccc1SCCc1c[nH]c2ccccc12] | 1 | | |
| 47 | 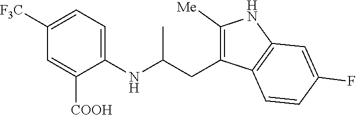 [SMILES: Cc1[nH]c2cc(F)ccc2c1CC(C)Nc1ccc(C(F)(F)F)cc1C(=O)O] | 0.17 | | |
| 48 | 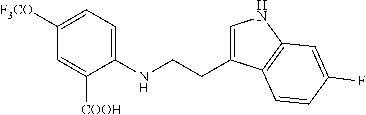 [SMILES: O=C(O)c1cc(OC(F)(F)F)ccc1NCCc1c[nH]c2cc(F)ccc12] | 0.18 | | |
| 49 | 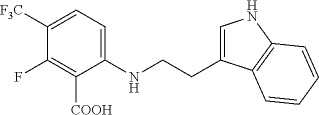 [SMILES: O=C(O)c1c(NCCc2c[nH]c3ccccc23)ccc(C(F)(F)F)c1F] | 1.4 | | |
| 50 | 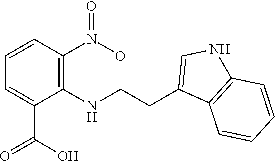 [SMILES: O=C(O)c1cccc([N+](=O)[O-])c1NCCc1c[nH]c2ccccc12] | 38 | | |
| 51 | 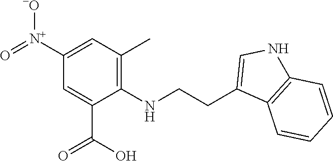 | 38 | | |
| 52 | 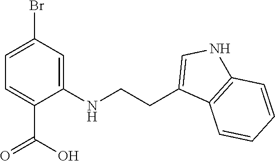 [SMILES: O=C(O)c1ccc(Br)cc1NCCc1c[nH]c2ccccc12] | 13 | | |
| 53 | 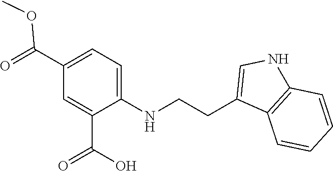 [SMILES: COC(=O)c1ccc(NCCc2c[nH]c3ccccc23)c(C(=O)O)c1] | 10 | | |
| 54 | 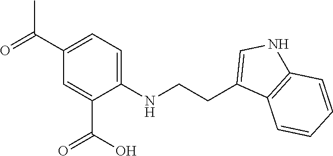 [SMILES: CC(=O)c1ccc(NCCc2c[nH]c3ccccc23)c(C(=O)O)c1] | 47 | | |
| 55 | 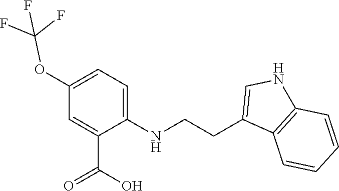 [SMILES: O=C(O)c1cc(OC(F)(F)F)ccc1NCCc1c[nH]c2ccccc12] | 0.94 | | |
| 56 | 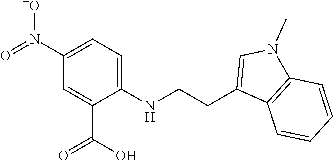 | 32 | | |
| 57 | 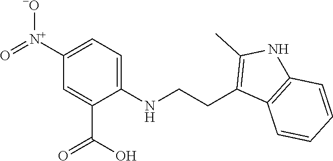 | 16 | | |
| 58 | 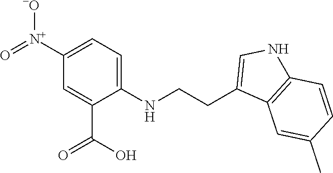 | 21 | | |
| 59 | 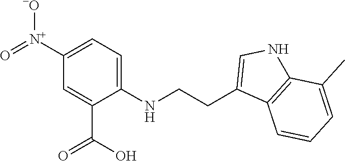 | 24 | | |
| 60 | 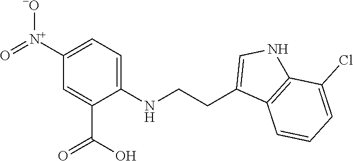 | 15 | | |
| 61 | 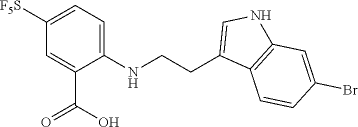 | 0.100 | | |
| 62 | 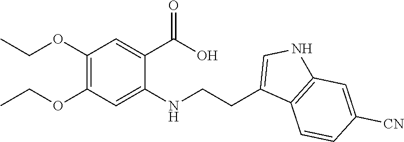 [SMILES: CCOc1cc(NCCc2c[nH]c3cc(C#N)ccc23)c(C(=O)O)cc1OCC] | 0.77 | | |
| 63 | 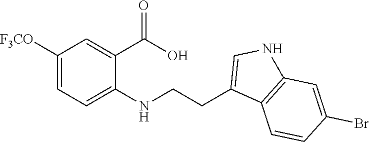 [SMILES: O=C(O)c1cc(OC(F)(F)F)ccc1NCCc1c[nH]c2cc(Br)ccc12] | <0.1 | | 0.01 |
| 64 | 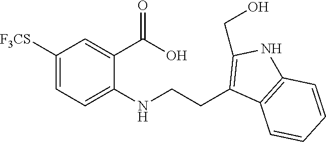 | 3.8 | | |
| 65 | 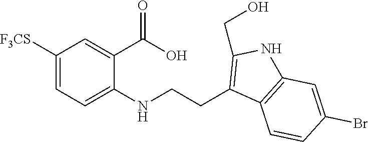 | 0.34 | | |
| 66 | 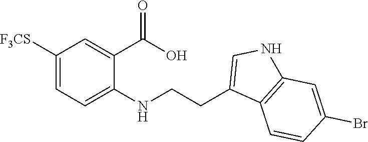 | 0.17 | | |
| 67 | 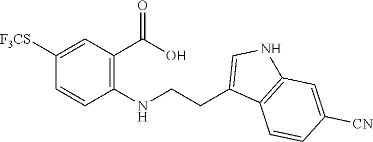 | <0.1 | | 0.0073 |
| 68 | 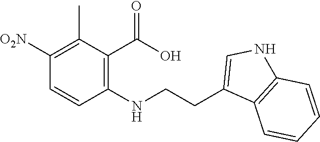 [SMILES: Cc1c([N+](=O)[O-])ccc(NCCc2c[nH]c3ccccc23)c1C(=O)O] | 4.9 | | |
| 69 | 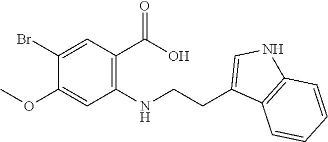 [SMILES: COc1cc(NCCc2c[nH]c3ccccc23)c(C(=O)O)cc1Br] | 28 | | |
| 70 | 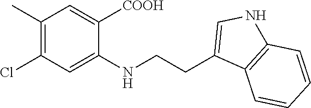 [SMILES: Cc1cc(C(=O)O)c(NCCc2c[nH]c3ccccc23)cc1Cl] | 11 | 6.3 | |
| 71 | 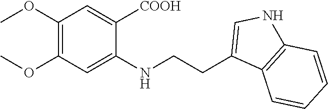 [SMILES: COc1cc(NCCc2c[nH]c3ccccc23)c(C(=O)O)cc1OC] | | 10 | |
| 72 | 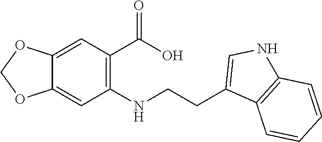 [SMILES: O=C(O)c1cc2c(cc1NCCc1c[nH]c3ccccc13)OCO2] | | | 8.3 |
| 73 | 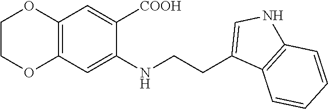 [SMILES: O=C(O)c1cc2c(cc1NCCc1c[nH]c3ccccc13)OCCO2] | | 30 | |
| 74 | 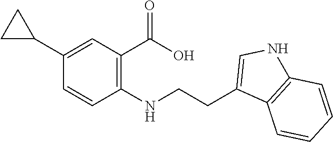 [SMILES: O=C(O)c1cc(C2CC2)ccc1NCCc1c[nH]c2ccccc12] | | | 6.2 |
| 75 | 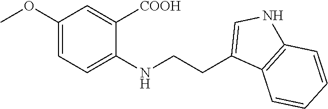 [SMILES: COc1ccc(NCCc2c[nH]c3ccccc23)c(C(=O)O)c1] | | 45 | |
| 76 | 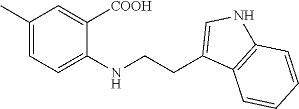 [SMILES: Cc1ccc(NCCc2c[nH]c3ccccc23)c(C(=O)O)c1] | | 36 | |
| 77 | 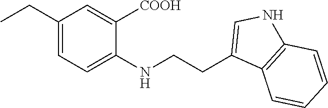 [SMILES: CCc1ccc(NCCc2c[nH]c3ccccc23)c(C(=O)O)c1] | 18 | 22 | |
| 78 | 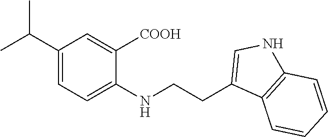 [SMILES: CC(C)c1ccc(NCCc2c[nH]c3ccccc23)c(C(=O)O)c1] | 30 | 8.9 | |
| 79 | 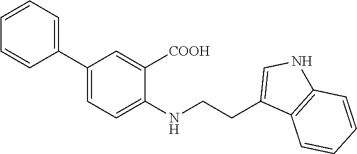 [SMILES: O=C(O)c1cc(-c2ccccc2)ccc1NCCc1c[nH]c2ccccc12] | 11 | >100 | |
| 80 | 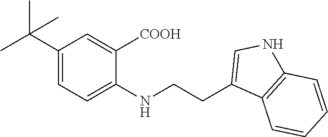 [SMILES: CC(C)(C)c1ccc(NCCc2c[nH]c3ccccc23)c(C(=O)O)c1] | 21 | >100 | |
| 81 | 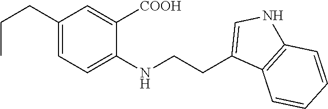 [SMILES: CCCc1ccc(NCCc2c[nH]c3ccccc23)c(C(=O)O)c1] | 5.6 | 6.5 | |
| 82 | 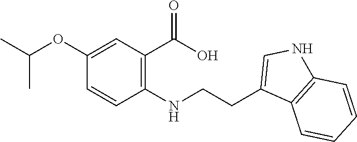 [SMILES: CC(C)Oc1ccc(NCCc2c[nH]c3ccccc23)c(C(=O)O)c1] | | | 7.7 |
| 83 | 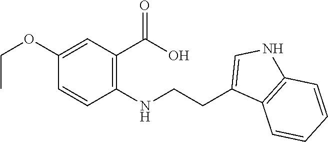 [SMILES: CCOc1ccc(NCCc2c[nH]c3ccccc23)c(C(=O)O)c1] | | | 9.1 |
| 84 | 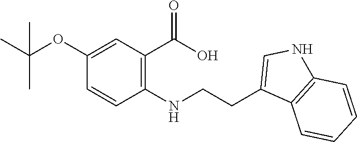 [SMILES: CC(C)(C)Oc1ccc(NCCc2c[nH]c3ccccc23)c(C(=O)O)c1] | | | 5.5 |
| 85 | 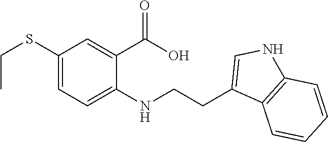 [SMILES: CCSc1ccc(NCCc2c[nH]c3ccccc23)c(C(=O)O)c1] | | 9.1 | |
| 86 | 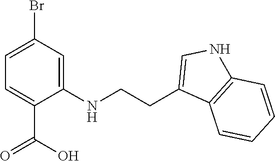 [SMILES: O=C(O)c1ccc(Br)cc1NCCc1c[nH]c2ccccc12] | 13 | | |
| 87 | 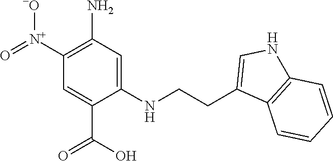 | 42 | | |
| 88 | 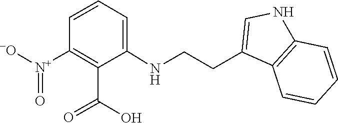 | 94 | 79 | |
| 89 | 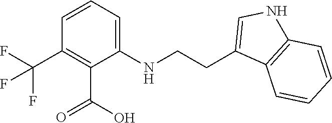 [SMILES: O=C(O)c1c(NCCc2c[nH]c3ccccc23)cccc1C(F)(F)F] | 120 | | |
| 90 | 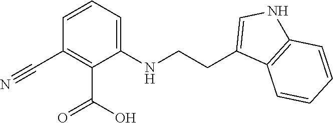 [SMILES: N#Cc1cccc(NCCc2c[nH]c3ccccc23)c1C(=O)O] | 37 | | |
| 91 | 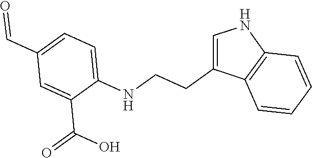 [SMILES: O=Cc1ccc(NCCc2c[nH]c3ccccc23)c(C(=O)O)c1] | 36 | | |
| 92 | 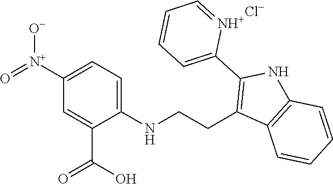 [SMILES: O=C(O)c1cc([N+](=O)[O-])ccc1NCCc1c(-c2cccc[nH+]2)[nH]c2ccccc12 ; [Cl-]] | 42 | | |
| 93 | 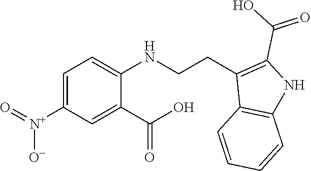 [SMILES: O=C(O)c1cc([N+](=O)[O-])ccc1NCCc1c(C(=O)O)[nH]c2ccccc12] | 16 | | |
| 94 | 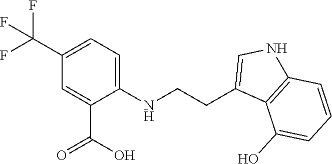 [SMILES: O=C(O)c1cc(C(F)(F)F)ccc1NCCc1c[nH]c2cccc(O)c12] | 51 | | |
| 95 | 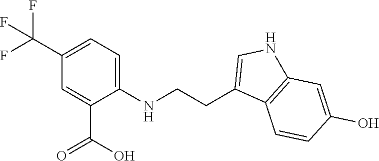 [SMILES: O=C(O)c1cc(C(F)(F)F)ccc1NCCc1c[nH]c2cc(O)ccc12] | 91 | | |

Example 97. The following illustrate representative pharmaceutical dosage forms, containing a

compound of Formula (I) (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X= (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of Formula (I): 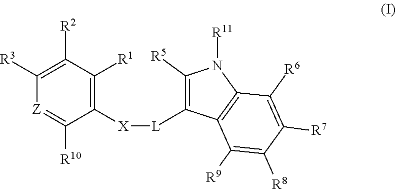
or a salt thereof, wherein:
R¹ is —COOR^{a} or tetrazol-5-yl;
R² is H, halo, nitro, trifluoromethyl, cyano, amino, (C₁-C₄)alkyl, or hydroxy;
R³ is H, nitro, cyano, halo, —SCF₃, —SF₅, (C₁-C₄)alkyl, (C₃-C₆)cycloalkyl, (C₁-C₄)alkoxycarbonyl, (C₁-C₄)alkanoyl, (C₁-C₄)alkoxy, phenyl, (C₁-C₄)alkylthio, trifluoromethyl, or trifluoromethoxy; or R³ and R^{x} taken together with the atoms to which they are attached form a 5-membered carboocycle, 6-membered carbocycle, a 5-membered heterocycle, a 6-membered heterocycle, or phenyl ring;
R⁵ is H, (C₁-C₄)alkyl, pyridyl, carboxy, or hydroxymethyl;
R⁶ is H, halo, (C₁-C₄)alkyl, or hydroxy;
R⁷ is H, halo, cyano, (C₁-C₄)alkyl, hydroxy, or (C₁-C₄)alkoxy, wherein any (C₁-C₄)alkyl and (C₁-C₄)alkoxy is optionally substituted with one or more fluoro;
R⁸ is H, halo, (C₁-C₄)alkyl, nitro, or cyano;
R⁹ is H, halo, hydroxy, or (C₁-C₄)alkyl;
R¹⁰ is H, halo, nitro, or (C₁-C₄)alkyl;
R¹¹ is H or (C₁-C₄)alkyl;
Z is CR^{x} or N;
R^{a} is H or (C₁-C₆)alkyl;
R^{x} is H, halo, nitro, (C₁-C₄)alkyl, amino, or (C₁-C₄)alkoxy; or R^{x} and R³ taken together with the atoms to which they are attached form a 5-membered carboocycle, 6-membered carbocycle, a 5-membered heterocycle, a 6-membered heterocycle, or phenyl ring;
X is S, O, or NH; and
L is absent or is (C₁-C₄)alkylene.

2. The compound or salt of claim 1, which is a compound of Formula (Ia): 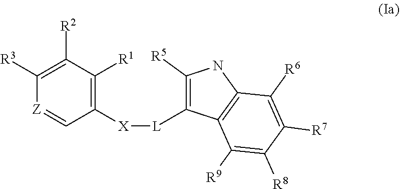
or a salt thereof, wherein:
R¹ is —COOR^{a} or tetrazol-5-yl;
R² is H, halo, amino, or hydroxy;
R³ is H, nitro, cyano, halo, —SCF₃, —SF₅, (C₁-C₄)alkyl, trifluoromethyl, or trifluoromethoxy; or R³ and R^{x} taken together with the atoms to which they are attached form a 6-membered carbocycle or phenyl ring;
R⁵ is H, (C₁-C₄)alkyl, or hydroxymethyl;
R⁶ is H, halo, or hydroxy;
R⁷ is H, halo, cyano, (C₁-C₄)alkyl, or (C₁-C₄)alkoxy, wherein any (C₁-C₄)alkyl and (C₁-C₄)alkoxy is optionally substituted with one or more fluoro;
R⁸ is H, halo, nitro, or cyano;
R⁹ is H, halo, or (C₁-C₄)alkyl;
Z is CR^{x} or N;
R^{a} is H or (C₁-C₆)alkyl;
R^{x} is H, halo, nitro, (C₁-C₄)alkyl, or (C₁-C₄)alkoxy; or R^{x} and R³ taken together with the atoms to which they are attached form a 6-membered carbocycle or phenyl ring;
X is S, O, or NH; and
L is absent or is (C₁-C₄)alkylene.

3. The compound or salt of claim 1, wherein R¹ is —COOR^{a}.

4. The compound or salt of claim 3, wherein R^{a} is H.

5. The compound or salt of claim 1, wherein R⁶ halo, (C₁-C₄)alkyl, or hydroxy.

6. The compound or salt of claim 1, wherein R⁷ is halo, cyano, (C₁-C₄)alkyl, hydroxy, or (C₁-C₄)alkoxy, wherein any (C₁-C₄)alkyl and (C₁-C₄)alkoxy is optionally substituted with one or more fluoro.

7. The compound or salt of claim 1, wherein R⁸ is halo, (C₁-C₄)alkyl, nitro, or cyano.

8. The compound or salt of claim 1, wherein Z is CR^{x} and R^{x} is halo, nitro, (C₁-C₄)alkyl, or (C₁-C₄)alkoxy.

9. The compound or salt of claim 1, wherein Z is N.

10. The compound or salt of claim 1, wherein X is NH.

11. The compound or salt of claim 1, wherein X is S.

12. The compound or salt of claim 1, wherein L is (C₂-C₄)alkylene.

13. The compound or salt of claim 1 that is selected from the group consisting of: 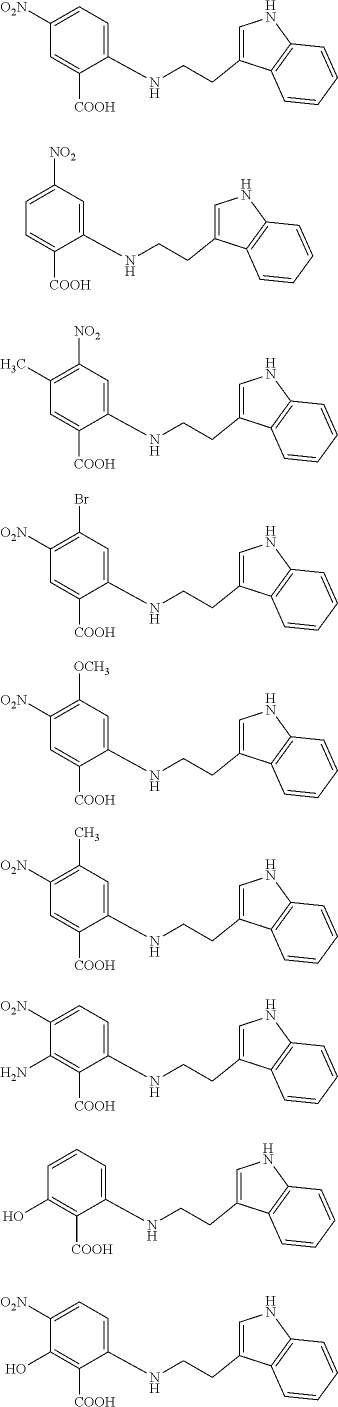 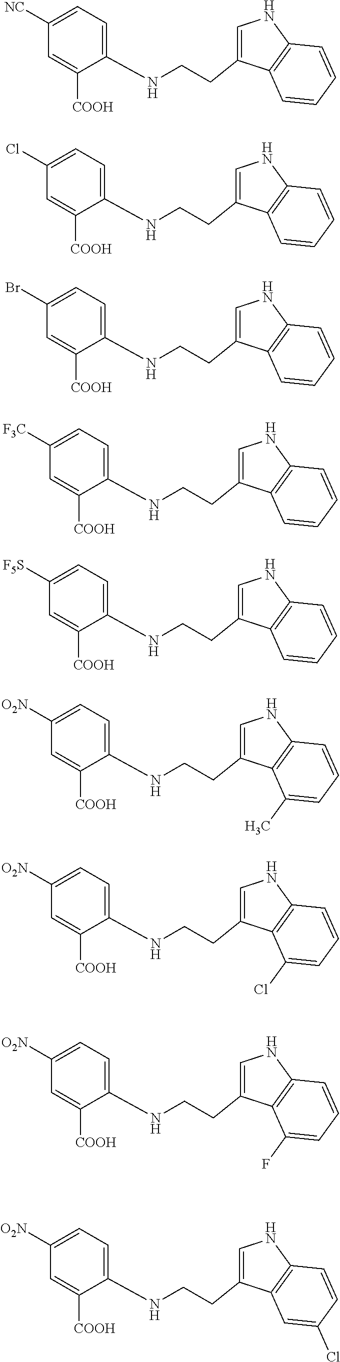 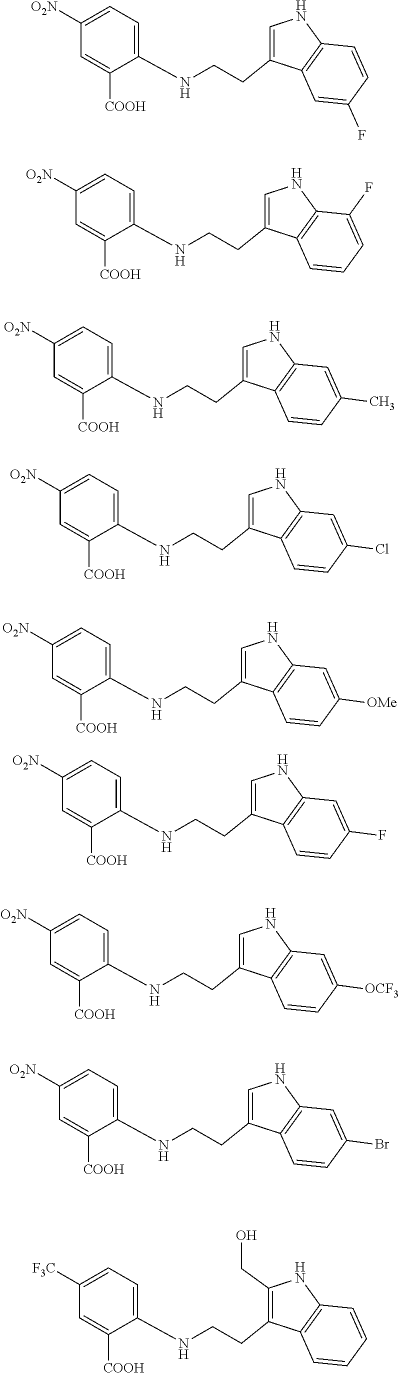 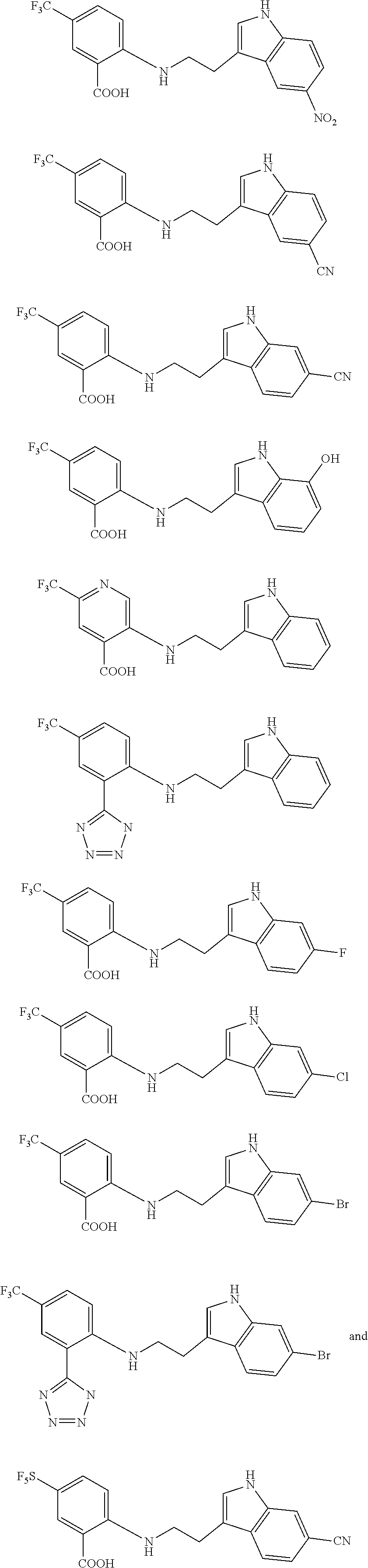
and salts thereof.

14. The compound or salt of claim 1 that is selected from the group consisting of: 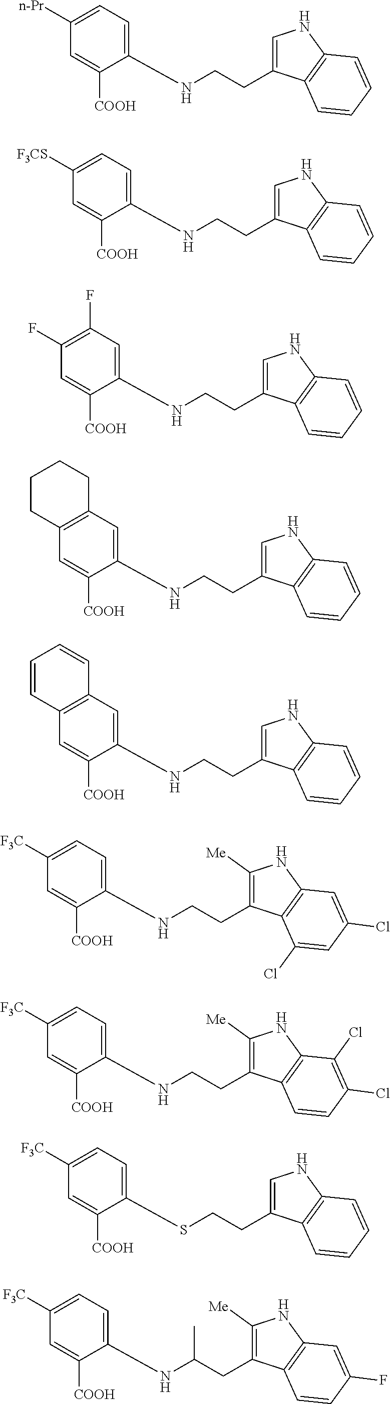 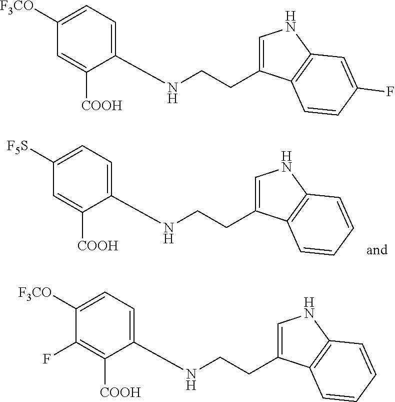
and salts thereof.

15. A pharmaceutical composition comprising a compound as described in claim 1 or a pharmaceutically acceptable salt thereof and a pharmaceutically acceptable excipient.

16. A method for treating cancer in an animal comprising, administering a compound of Formula (I) as described in claim 1 or a pharmaceutically acceptable salt thereof to the animal.

17. A method for producing a contraceptive effect in an animal comprising, administering a compound as described in claim 1 or a pharmaceutically acceptable salt thereof to the animal.

18. A method to reduce sperm count in a male subject comprising, administering a compound as described in claim 1 or a pharmaceutically acceptable salt thereof to the male subject.

19. A method to produce reversible infertility in a male subject comprising, administering a compound as described in claim 1 or a pharmaceutically acceptable salt thereof to the male subject.

20. A method to reduce the likelihood of conception following intercourse between a male subject and a female subject comprising, administering a compound as described in claim 1 or a pharmaceutically acceptable salt thereof to the male subject prior to the intercourse.
